(19) United States
(12) Patent Application Publication
(10) Pub. No.: US 2016/0369006 A1
(43) Pub. Date: Dec. 22, 2016

(54) Fc-ENHANCED ANTI-WT1/HLA ANTIBODY

(71) Applicants: MEMORIAL SLOAN-KETTERING CANCER CENTER, New York, NY (US); EUREKA THERAPEUTICS, INC., Emeryville, CA (US); NOVARTIS AG, Basel (CH)

(72) Inventors: David SCHEINBERG, New York, NY (US); Nicholas VEOMETT, New York, NY (US); Hong LIU, El Sobrante, CA (US); Jingyi XIANG, Walnut Creek, CA (US); Cheng LIU, Oakland, CA (US); Tao DAO, New York, NY (US); Heather Adkins HUET, Cambridge, MA (US)

(73) Assignees: Memorial Sloan-Kettering Cancer Center, New York, NY (US); Eureka Therapeutics, Inc., Emeryville, CA (US)

(21) Appl. No.: 15/101,273

(22) PCT Filed: Nov. 7, 2014
(86) PCT No.: PCT/US2014/064657

§ 371 Date: Jun. 2, 2016

Related U.S. Application Data
(60) Provisional application No. 61/901,210, filed on Nov. 7, 2013.

(51) Int. Cl. C07K 16/32; C07K 16/30

(52) U.S. Cl. CPC C07K 16/32; C07K 16/3069; C07K 16/3046; C07K 16/3015; C07K 2317/565; C07K 2317/34; C07K 2317/41; C07K 2317/92; C07K 2317/732; A61K 2039/505

(57) ABSTRACT

The present disclosure relates to an anti-WT-1/HLA/A2 antibody with enhanced antibody dependent cell-mediated cytotoxicity (ADCC) function due to altered Fc glycosylation. The antibody, which has reduced fucose and/or galactose, was compared to its normally glycosylated counterpart in binding assays, in vitro ADCC assays, and mesothelioma and leukemia therapeutic models and pharmacokinetic studies in mice. The antibody with normal glycosylation mediated ADCC against hematopoietic and solid tumor cells at concentrations below 1 μg/ml, but the reduced fucosylated antibody was about 5-10 fold more potent in vitro against multiple cancer cell lines, was more potent in vivo against JMN mesothelioma, and effective against SET2 AML and fresh ALL xenografts. ESKM had a shortened half-life (4.9 vs 6.5 days), but an identical biodistribution pattern in C57BL6/J mice. At therapeutic doses of ESKM, there was no difference in half-life or biodistribution in HLA-A2.1+ transgenic mice compared to the parent strain. Importantly, therapeutic doses of ESKM in these mice caused no depletion of total WBCs or hematopoietic stem cells, or pathologic tissue damage.

### CROSS-REFERENCE TO RELATED APPLICATIONS

The benefit under 35 U.S.C. §119(e) of U.S. Provisional Patent Application Ser. No. 61/901,210 filed Nov. 7, 2013, is hereby claimed, and the disclosure of the priority document is incorporated herein by reference in its entirety.

This application contains subject matter that is related to the subject matter of U.S. Provisional Application No. 61/470,635, filed Apr. 1, 2011, U.S. Provisional Application No. 61/491,392 filed May 31, 2011 and U.S. application Ser. No. 14/008,447, which is a national stage entry of PCT International Application No. PCT/US2012/031892 filed Apr. 1, 2012. These applications are hereby incorporated by reference in their entirety into the present disclosure.

### STATEMENT OF RIGHTS UNDER FEDERALLY-SPONSORED RESEARCH

This invention was made with government support under grants P01CA23766, R01CA55349 and T32 CA062948 awarded by the U.S. National Institutes of Health. The government has certain rights in the invention.

### SEQUENCE LISTING

This application contains a Sequence Listing, created on Nov. 7, 2014; the file, in ASCII format, is designated 48316_SeqListing.txt and is 46,083 bytes in size. The file is hereby incorporated by reference in its entirety into the application

### TECHNICAL FIELD

The present invention relates generally to antibodies against cytosolic proteins. More particularly, the invention relates to antibodies against Wilm's tumor oncogene protein (WT1), specifically antibodies that recognize a WT1 peptide in conjunction with a major histocompatibility antigen.

### BACKGROUND OF THE INVENTION

Therapeutic monoclonal antibodies (mAbs) are highly specific and effective drugs, with pharmacokinetics suitable for infrequent dosing. However, all current marketed therapeutic anticancer mAbs target extracellular or cell-surface molecules, whereas many of the most important tumor-associated and oncogenic proteins are nuclear or cytoplasmic (Sensi M and Anichini A. Clinical cancer research: an official journal of the American Association for Cancer Research 2006; 12(17):5023-32; Kessler J H and Melief C J. Leukemia 2007; 21(9):1859-74).

Intracellular proteins are processed by the proteasome and presented on the cell surface as small peptides in the pocket of major histocompatibility complex (MHC) class I molecules (in humans, also called human leukocyte antigen, HLA) allowing recognition by T-cell receptors (TCRs) (Morris E et al. Blood Rev 2006; 20(2):61-9; Konig R. Curr Opin Immunol 2002; 14(1):75-83). Therefore, mAbs that mimic the specificity of TCRs (that is, recognizing a peptide presented in the context of a specific HLA-type) can bind cell-surface complexes with specificity for an intracellular protein. A “TCR-mimic” (TCRm) antibody was first reported by Andersen et. al. (Andersen P S et al. Proceedings of the National Academy of Sciences of the United States of America 1996; 93(5):1820-4), and several have since been developed by various groups (Epel M et al. European journal of immunology 2008; 38(6):1706-20; Wittman V P et al. J Immunol 2006; 177(6):4187-95; Klechevsky et al. Cancer research 2008; 68(15):6360-7; Bhattacharya R et al. Journal of cellular physiology 2010; 225(3):664-72; Verma B, et al. J Immunol 2010; 184(4):2156-65; Sergeeva et al. Blood 2011; 117(16):4262-72).

The first fully human TCRm mAb, called ESK1, that specifically targets RMFPNAPYL (RMF), a peptide derived from Wilms' tumor gene 1 (WT1), presented in the context of HLA-A0201 was recently reported (Dao T et al. Science translational medicine 2013; 5(176):176ra33). WT1 is an important, immunologically validated oncogenic target that has been the focus of many vaccine trials (Dao T et al. Best practice & research Clinical haematology 2008; 21(3):391-404). WT1 is a zinc finger transcription factor with limited expression in normal adult tissues, but is over expressed in the majority of leukemias and a wide range of solid tumors (Sugiyama H. Japanese journal of clinical oncology 2010; 40(5):377-87). WT1 was ranked as the top cancer antigenic target for immunotherapy by a National Institutes of Health-convened panel (Cheever M A et al. Clinical cancer research: an official journal of the American Association for Cancer Research 2009; 15(17):5323-37); further, WT1 expression is a biomarker and a prognostic indicator in leukemia (Inoue K et al. Blood 1994; 84(9):3071-9, Ogawa H et al. Blood 2003; 101(5):1698-704). ESK1 mAb specifically bound to leukemias and solid tumor cell lines that are both WT1+ and HLA-A0201+ and showed efficacy in mouse models in vivo against several WT1+ HLA-A0201+ leukemias (Dao T et al. Science translational medicine 2013; 5(176):176ra33). Therefore, ESK1 is a useful therapeutic platform for further clinical development, and improvements to the native antibody could help potentiate its effect and improve clinical efficacy.

### SUMMARY OF THE INVENTION

In one aspect, the invention relates to an antibody comprising: (A) a heavy chain (HC) variable region comprising HC-CDR1, HC-CDR2 and HC-CDR3 respectively, comprising amino acid sequences SEQ ID NOS: 2, 3, and 4; 18, 19 and 20; 34, 35, and 36; 50, 51, and 52; 66, 67, and 68 or 82, 83, and 84; and a light chain (LC) variable region comprising LC-CDR1, LC-CDR2 and LC-CDR3 respectively, comprising amino acid sequences SEQ ID NOS: 8, 9 and 10; 24, 25 and 26; 40, 41 and 42; 56, 57 and 58; 72, 73 and 74 or 88, 89 and 90; or (B) a VH and VL comprising the amino acid sequence of SEQ ID NO: 14 and SEQ ID NO: 16; 30 and 32; 46 and 48; 62 and 64; 78 and 80 or 94 and 96, respectively, wherein said antibody has no detectable fucose or galactose. As a result of the modification in glycosylation, the altered antibody exhibits between 50-100% (80%) higher affinity for activating human FcγRIIIa (158V variant) than normally glycosylated antibody, has 3-4-fold (3.5-fold) higher affinity for a FcγRIIIa 158F variant than normally glycosylated antibody, and has between 30 and 70% (50%) reduced affinity for inhibitory FcγRIIb than normally glycosylated antibody.

In one embodiment, the antibody comprises a light chain consisting essentially of the amino acid sequence of SEQ ID NO: 100 and a heavy chain consisting essentially of the amino acid sequence of SEQ ID NO: 101.

In a related aspect, the invention relates to isolated nucleic acids, vectors and cells comprising a nucleic acid that encodes an antibody as described herein.

In yet another aspect, the invention relates to the use of an antibody as disclosed herein for the treatment of a WT1 positive disease and therefore, to a pharmaceutical composition comprising an antibody of the invention and a pharmaceutically acceptable carrier.

In a related aspect, the invention relates to a method for treatment of a subject having a WT1-positive disease, comprising administering to the subject a therapeutically effective amount of an antibody disclosed herein. WT1-positive disease amenable to treatment with the antibody of the invention includes chronic leukemia or acute leukemia or WT1+ cancer, including, for example, chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma.

The foregoing summary is not intended to define every aspect of the invention, and other features and advantages of the present disclosure will become apparent from the following detailed description, including the drawings. The present disclosure is intended to be related as a unified document, and it should be understood that all combinations of features described herein are contemplated, even if the combination of features are not found together in the same sentence, paragraph, or section of this disclosure. In addition, the disclosure includes, as an additional aspect, all embodiments of the invention narrower in scope in any way than the variations specifically mentioned above. With respect to aspects of the disclosure described or claimed with “a” or “an,” it should be understood that these terms mean “one or more” unless context unambiguously requires a more restricted meaning. With respect to elements described as one or more within a set, it should be understood that all combinations within the set are contemplated. If aspects of the disclosure are described as “comprising” a feature, embodiments also are contemplated “consisting of” or “consisting essentially of” the feature. Additional features and variations of the disclosure will be apparent to those skilled in the art from the entirety of this application, and all such features are intended as aspects of the disclosure.

### BRIEF DESCRIPTION OF THE DRAWINGS

FIG. 1 shows that ESKM has a modified Fc glycosylation pattern, altering binding to FcγRs but not to the RMF/A2 target. (1A) Comparison of the oligosaccharide profile of ESK1 and ESKM. Peak assignment is based on the retention time and the monosaccharide composition analysis. G# indicates the number of terminal galactoses (0, 1, or 2), F indicates presence of core fucose, Hex5GlcNAc2 denotes (GlcNAc)2 core with terminal Hexose 5 glycan structure (terminating in mannose and/or glucose). (1B) Summary of ESK1 and ESKM binding to mouse and human FcγRs. Anti-mouse FcR binding was assessed by ELISA, while anti-human FcR binding was determined by FCM titration on FcγR-expressing CHO cells. Representative binding curves of ESK1 and ESKM against human FcRn (1C), mouse FcγRIV (1D), and mouse FcγRIIb (1E). ¹²⁵I-labeled ESK1 (1F) and ESKM (1G) mAbs were titrated against JMN cells. All curves were fit with a non-linear single-site total binding saturation curve, and K_{d} was calculated using Prism software. ESKM having 100% reduced fucose content relative to ESK1 wildtype IgG1 showed improved reverse signaling through FcγRIIIa compared to ESK1 wildtype IgG1 and ESK1 containing D265A/P329A mutations in the Fc domain (ESK1-DAPA) (1H).

FIG. 2 shows that ESKM is more efficacious and potent in ADCC assays with human PBMC effectors at the indicated mAb concentrations and effector/target (E:T) ratios. Cytotoxicity was measured by 4-hour ⁵¹Cr release assay. (2A) T2 cells were pulsed with RMF peptide and incubated with 3 μg/mL mAb. HLA-A0201+ human leukemia cell lines: (2B) BA25 ALL, (2C) AML14 and (2D) SET2 AML (2E) HLA-A0201 negative HL60 promyelocytic leukemia. HLA-A0201+ human mesothelioma cell lines: (2F) JMN, (2G) Meso37 and (2H) Meso56. Data presented are averages of triplicate measurements from representative experiments, all with isolated PBMCs from the same donor. All cell lines, with exception of Meso37 and Meso56, were repeated 3 or more times with multiple donors. Both ESKM having 100% reduced fucose content relative to ESK1 wildtype IgG1 and ESKM having 70% reduced fucose content relative to ESK1 wildtype IgG1 resulted in greater ADCC killing of OV56 ovarian cancer cells compared to ESK1 wildtype IgG1 and ESK1 containing D265A/P329A mutations in the Fc domain (ESK1-DAPA) (2I).

FIG. 3 shows that ESKM more effectively treats JMN mesothelioma in SCID mice. Tumor burden was determined by luciferase imaging of mice in the supine position (n=5 per group). Where noted, signal was normalized to the day 4 signal for each mouse. Arrows indicate treatment with mAb. (3A) ESKM significantly reduced mean tumor growth as assessed by total luminescence (*p<0.05, multiple T-tests on and after day 18). (3B) ESKM reduced individual tumor burden during the treatment course in 3 of 5 mice. This effect was reproduced in a second experiment in the same model. (3C) ESKM also significantly improved survival (p=0.016 vs isotype, p=0.095 vs ESK1), with events representing death or terminal morbidity as assessed on protocol by veterinarians. (3D) ESKM is effective against SET2 AML (*p<0.05, multiple T-tests). (3E-3F) ESKM is effective against a disseminated fresh, patient-derived pre-B ALL (*p<0.05, **p<0.01, multiple T-tests). (3G) Bone marrow cells were harvested from mice in F, and transplanted as subcutaneous tumors into NSG mice. Bone marrow cells from the isotype-treated mice were injected into the right shoulder flank (viewed from above), while an equal number of bone marrow cells from the ESKM-treated mice were injected into the left shoulder. (3H) Quantitation of total bone-marrow signal from mice in 3F, before harvesting. (3I) Quantitation of subcutaneous tumors in 3G, 28 days post transplantation.

FIG. 4 shows that ESKM and native ESK1 display similar pharmacokinetics and biodistribution. ¹²⁵I-labeled mAb was injected IV and activity was measured in blood or harvested organs (n=3 per group). Pharmacokinetics (4A) and biodistribution (4B) of ESKM or native ESK1 (3 μg each) in C57BL6/J mice. (4C) Pharmacokinetics of ESKM (2 μg) in C57BL6/J or HLA-A0201+ transgenic mice. (4D) Biodistribution of ESKM (100 μg) in C57BL6/J or HLA-A0201+ transgenic mice, harvested after 1 day. ESKM (4E) or hIgG1 isotype control (4F) (2 μg each) in C57BL6/J or HLA-A0201+ transgenic mice, harvested after 1 day.

FIG. 5 shows that ESKM treatment does not affect leukocyte or hematopoietic stem cell (HSC) counts in HLA-A0201+ transgenic mice. Animals (n=5 per group) were treated with 100 μg ESKM or hIgG1 isotype control on days 0 and 4; blood and bone marrow were harvested on day 5. (5A) Total white blood cell (WBC) and WBC subset cell counts. Absolute number (5B) and frequency (5C) of lineage-SCA1+ KIT+ (LSK) cells. Absolute number (5D) and frequency (5E) of long-term HSCs (Slamf1+CD34− LSK cells).

FIG. 6 shows that ESKM has no significant effect against intraperitoneal JMN mesothelioma in NOG mice. Mice were engrafted intraperitoneally with 3×10⁶ luciferase+ JMN cells, then treated with 50 μg ESKM or hIgG1 isotype control antibody twice weekly starting on day 4 via intraperitoneal injections.

FIG. 7 shows that all human antibodies tested accumulated more in spleens of HLA-A2+ transgenic mice, but ESK1 did not bind specifically to isolated HLA-A2+ spleen, bone marrow or thymus cells. (7A) Accumulation of ¹²⁵I-labeled antibodies in spleens of C57BL6/J or HLA-A2+ transgenic mice relative to antibody level in the blood. Mice were injected retroorbitally with 2 μg indicated antibody, then sacrificed after 24 hours for blood and spleen collection. (7B) Specific binding of ¹²⁵I-labeled ESK1 to bone marrow, spleen, or thymus cells isolated from C57BL6/J or HLA-A2+ transgenic mice. Tissues were collected from 2 (C57) or 3 (HLA-A2+ transgenic) mice, then bound by 1 μg/mL ¹²⁵I-labeled ESK1 either alone or after blocking with 50-fold excess unlabeled ESK1. Specific binding was determined, and #ESK1 bound per cell was calculated.

### DETAILED DESCRIPTION OF THE INVENTION

All publications, patents and other references cited herein are incorporated by reference in their entirety into the present disclosure.

Unless otherwise defined herein, scientific and technical terms used in connection with the present invention shall have the meanings that are commonly understood by those of ordinary skill in the art. Further, unless otherwise required by context, singular terms shall include pluralities, and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. In practicing the present invention, many conventional techniques in molecular biology, microbiology, cell biology, biochemistry, and immunology are used, which are within the skill of the art. These techniques are described in greater detail in, for example, Molecular Cloning: a Laboratory Manual 3rd edition, J. F. Sambrook and D. W. Russell, ed. Cold Spring Harbor Laboratory Press 2001; Recombinant Antibodies for Immunotherapy, Melvyn Little, ed. Cambridge University Press 2009; “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001). The contents of these references and other references containing standard protocols, widely known to and relied upon by those of skill in the art, including manufacturers' instructions are hereby incorporated by reference as part of the present disclosure. The following abbreviations are used throughout the application:

Ab: Antibody

ADCC: Antibody-dependent cellular cytotoxicity

ALL: Acute lymphocytic leukemia

AML: Acute myeloid leukemia

CDC: Complement dependent cytotoxicity

CMC: Complement mediated cytotoxicity

CDR: Complementarity determining region (see also HVR below)

CL: Constant domain of the light chain

CH1: 1st constant domain of the heavy chain

CH1, 2, 3: 1st, 2nd and 3rd constant domains of the heavy chain

CH2, 3: 2nd and 3rd constant domains of the heavy chain

CHO: Chinese hamster ovary

CTL: Cytotoxic T cell

EC50: Half maximal effective concentration

E:T Ratio: Effector:Target ratio

Fab: Antibody binding fragment

FACS: Flow assisted cytometric cell sorting

FBS: Fetal bovine serum

FR: Framework region

HC: Heavy chain

HLA: Human leukocyte antigen

HVR-H: Hypervariable region-heavy chain (see also CDR)

HVR-L: Hypervariable region-light chain (see also CDR)

Ig: Immunoglobulin

IRES: Internal ribosome entry site

Ko: Dissociation constant

k_{off}: Dissociation rate

kₒₙ: Association rate

MHC: Major histocompatibility complex

MM: Multiple myeloma

VH: Variable heavy chain includes heavy chain hypervariable region and heavy chain variable framework region

VL: Variable light chain includes light chain hypervariable region and light chain variable framework region

WT1: Wilms tumor protein 1

In the description that follows, terms used herein are intended to be interpreted consistently with the meaning of those terms as they are known to those of skill in the art. The definitions provided herein below are meant to clarify, but not limit, the terms defined.

As used herein, “administering” and “administration” refer to the application of an active ingredient to the body of a subject.

“Antibody” and “antibodies” as those terms are known in the art refer to antigen binding proteins of the immune system. The term “antibody” as referred to herein includes whole, full length antibodies having an antigen-binding region, and any fragment thereof in which the “antigen-binding portion” or “antigen-binding region” is retained, or single chains, for example, single chain variable fragment (scFv), thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant (CH) region. The heavy chain constant region is comprised of three domains, CH1, CH2 and CH3. Each light chain is comprised of a light chain variable region (abbreviated herein as VL) and a light chain constant CL region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” or “antigen-binding region” of an antibody, as used herein, refers to that region or portion of the antibody that binds to the antigen and which confers antigen specificity to the antibody; fragments of antigen-binding proteins, for example, antibodies includes one or more fragments of an antibody that retain the ability to specifically bind to an antigen (e.g., an peptide/HLA complex). It has been shown that the antigen-binding function of an antibody can be performed by fragments of a full-length antibody. Examples of antigen-binding fragments encompassed within the term “antibody fragments” of an antibody include a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., Nature 1989; 341:544-546), which consists of a VH domain; and an isolated complementarity determining region (CDR).

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules. These are known as single chain Fv (scFv); see e.g., Bird et al., 1988 Science 242:423-426; and Huston et al., 1988 Proc. Natl. Acad. Sci. 85:5879-5883. These antibody fragments are obtained using conventional techniques known to those of skill in the art, and the fragments are screened for utility in the same manner as are intact antibodies.

As used herein, the term “effective amount” means that amount of a compound or therapeutic agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The present disclosure provides compositions and methods of treatment relating to recombinant antibodies. TCRm antibodies are potentially limited by the extremely low number of epitopes presented on the target cell, which may be as few as several hundred sites (Dao T et al. Science translational medicine 2013; 5(176):176ra33). Therefore, mechanisms to enhance potency may be essential to their success in humans as therapeutic agents against cancer.

The mechanisms of action of mAbs can be enhanced through Fc region protein engineering (Desjarlais J R et al. Drug discovery today 2007; 12(21-22):898-910), or by modification of Fc-region glycosylation (Jefferis R. Biotechnology progress 2005; 21(1):11-6; Hodoniczky J et al. Biotechnology progress 2005; 21(6):1644-52). Removal of fucose from the carbohydrate chain increases mAb binding affinity for the activating FcγRIIIa receptor and enhances ADCC (de Romeuf C et al. British journal of haematology 2008; 140(6):635-43; Masuda K, et al. Molecular immunology 2007; 44(12):3122-31; Shields R L et al. The Journal of biological chemistry 2002; 277(30):26733-40; Shinkawa T et al. The Journal of biological chemistry 2003; 278(5):3466-73). The addition of bisecting N-acetyl-D-glucosamine (GlcNAc) can also significantly enhance ADCC (Shinkawa T et al. The Journal of biological chemistry 2003; 278(5):3466-73; Davies J et al. Biotechnology and bioengineering 2001; 74(4):288-94; Umana P et al. Nature biotechnology 1999; 17(2):176-80). However, removal or replacement of the terminal galactose residues present on endogenous IgG reduces complement dependent cytotoxicity (CDC) activity (Hodoniczky J et al. Biotechnology progress 2005; 21(6):1644-52; Boyd P N et al. Molecular immunology 1995; 32(17-18):1311-8).

An Fc-modified antibody can be generated by expressing a construct encoding an anti-WT1/HLA/A2 antibody, for example, as disclosed in WO 2012/135854, in MAGE 1.5 CHO cells in accordance with methodology disclosed in U.S. Pat. No. 8,025,879 (Eureka Therapeutics, Inc), resulting in a consistent pattern of defucosylation and exposed terminal hexose (mannose and/or glucose), allowing higher affinity for activating human FcγRIIIa and murine FcγRIV while decreasing affinity for inhibitory FcγRIIb. A modified antibody of the present disclosure, designated herein as “ESKM”, has reduced fucose content and/or galactose content, e.g., relative to a wild-type antibody. The fucose content and/or galactose content can be reduced by 30% to 100% using any method known in the art.

ESKM mediated ADCC at lower doses than native ESK1 and was more potent in human tumor models in vivo. Further, ESKM had similar pharmacokinetics and biodistribution to the native antibody. ESKM showed no observable off-target tissue sink in wild-type mice, and at therapeutic doses there was no difference in half-life or biodistribution in HLA-A2.1+ transgenic mice compared to the parent strain. Importantly, therapeutic doses of ESKM in these mice caused no depletion of total WBCs or hematopoietic stem cells (HSCs), or pathologic tissue damage. The retained specificity, enhanced potency, favorable pharmacokinetics and distribution, and lack of toxicity in these models support ESKM as to treat a wide variety of cancers and leukemias.

In one embodiment, the antibody of the invention is an anti-WT1/HLA-A2 antibody having an antigen binding region that specifically binds to a WT1 peptide with the amino acid sequence RMFPNAPYL (SEQ ID NO: 1) in conjunction with HLA-A0201.

In some embodiments, the antibody of the invention comprises one of the combinations of amino acid sequences for CDRs, and heavy and light chain variable regions from Tables 1-6.

TABLE 1

| Antigen | WT1 (Ext002 #3) | | |
|---|---|---|---|
| Peptide | RMFPNAPYL (SEQ ID NO: 1) | | |
| CDRs: | 1 | 2 | 3 |
| VH | GGTFSSYAIS | GIIPIFGTANYAQKFQG | RIPPYYGMDV |
| | (SEQ ID NO: 2) | (SEQ ID NO: 3) | (SEQ ID NO: 4) |
| DNA | ggaggcaccttcagcag | gggatcatccctatctttggtac | cggattcccccgtactacggtat |
| | ctatgctatcagc | agcaaactacgcacagaagtt | ggacgtc (SEQ ID NO: 7) |
| | (SEQ ID NO: 5) | ccagggc | |
| | | (SEQ ID NO: 6) | |
| VL | SGSSSNIGSNYVY | RSNQRPS | AAWDDSLNGVV |
| | (SEQ ID NO: 8) | (SEQ ID NO: 9) | (SEQ ID NO: 10) |
| DNA | tctggaagcagctccaac | aggagtaatcagcggccctca | gcagcatgggatgacagcctg |
| | atcggaagtaattatgtat | (SEQ ID NO: 12) | aatggtgtggta |
| | ac (SEQ ID NO: 11) | | (SEQ ID NO: 13) |

| | |
|---|---|
| Full | QVQLVQSGAEVKKPGSSVKVSCKASGGTFSSYAISWVRQAPGQGLE |
| VH | WMGGIIPIFGTANYAQKFQGRVTITADESTSTAYMELSSLRSEDTAVYY |
| | CARRIPPYYGMDVWGQGTTVTVSS (SEQ ID NO: 14) |
| DNA | caggtgcagctggtgcagtctggggctgaggtgaagaagcctgggtcctcggtgaaggtctcctgc |
| | aaggcttctggaggcaccttcagcagctatgctatcagctgggtgcgacaggcccctggacaagg |
| | gcttgagtggatgggagggatcatccctatctttggtacagcaaactacgcacagaagttccaggg |
| | cagagtcacgattaccgcggacgaatccacgagcacagcctacatggagctgagcagcctgag |
| | atctgaggacacggccgtgtattactgtgcgagacggattcccccgtactacggtatggacgtctgg |
| | ggccaagggaccacggtcaccgtctcctca (SEQ ID NO: 15) |
| Full | QTVVTQPPSASGTPGQRVTISCSGSSSNIGSNYVYWYQQLPGTAPKL |
| VL | LIYRSNQRPSGVPDRFSGSKSGTSASLAISGPRSVDEADYYCAAWDD |
| | SLNGVVFGGGTKLTVLG (SEQ ID NO: 16) |
| DNA | cagactgtggtgactcagccaccctcagcgtctgggacccccgggcagagggtcaccatctcttgtt |
| | ctggaagcagctccaacatcggaagtaattatgtatactggtaccaacagctcccaggaacggcc |
| | cccaaactcctcatctataggagtaatcagcggccctcaggggtccctgaccgattctctggctcca |
| | agtctggcacctcagcctccctggccatcagtgggccccggtccgtggatgaggctgattattactgt |
| | gcagcatgggatgacagcctgaatggtgtggtattcggcggagggaccaagctgaccgtcctagg |
| | t (SEQ ID NO: 17) |

TABLE 2

| Antigen | WT1 (Ext002 #5) | | |
|---|---|---|---|
| Peptide | RMFPNAPYL (SEQ ID NO: 1) | | |
| CDRs | 1 | 2 | 3 |
| VH | GDSVSSNSAAWN | RTYYGSKWYNDYAVS | GRLGDAFDI |
| | (SEQ ID NO: 18) | VKS (SEQ ID NO: 19) | (SEQ ID NO: 20) |
| DNA | ggggacagtgtctctagc | aggacatactacgggtccaag | ggtcgcttaggggatgcttttga |
| | aacagtgctgcttggaac | tggtataatgattatgcagtatct | tatc (SEQ ID NO: 23) |
| | (SEQ ID NO: 21) | gtgaaaagt (SEQ ID NO: | |
| | | 22) | |
| VL | RASQSISSYLN | AASSLQS | QQSYSTPLT |
| | (SEQ ID NO: 24) | (SEQ ID NO: 25) | (SEQ ID NO: 26) |
| DNA | cgggcaagtcagagcatt | gctgcatccagtttgcaaagt | caacagagttacagtacccct |
| | agcagctatttaaat | (SEQ ID NO: 28) | ctcact (SEQ ID NO: 29) |
| | (SEQ ID NO: 27) | | |

| | |
|---|---|
| Full | QVQLQQSGPGLVKPSQTLSLTCAISGDSVSSNSAAWNWIRQSPSRGL |
| VH | EWLGRTYYGSKWYNDYAVSVKSRITINPDTSKNQFSLQLNSVTPEDTA |
| | VYYCARGRLGDAFDIWGQGTMVTVSS (SEQ ID NO: 30) |
| DNA | caggtacagctgcagcagtcaggtccaggactggtgaagccctcgcagaccctctcactcacctgt |
| | gccatctccggggacagtgtctctagcaacagtgctgcttggaactggatcaggcagtccccatcg |
| | agaggccttgagtggctgggaaggacatactacgggtccaagtggtataatgattatgcagtatctg |
| | tgaaaagtcgaataaccatcaacccagacacatccaagaaccagttctccctgcagctgaactct |
| | gtgactcccgaggacacggctgtgtattactgtgcaagaggtcgcttaggggatgcttttgatatctgg |
| | ggccaagggacaatggtcaccgtctcttca (SEQ ID NO: 31) |
| Full | DIQMTQSPSSLSASVGDRVTITCRASQSISSYLNWYQQKPGKAPKLLIY |
| VL | AASSLQSGVPSRFSGSGSGTDFTLTISSLQPEDFATYYCQQSYSTPLT |
| | FGGGTKVDIKR (SEQ ID NO: 32) |
| DNA | gacatccagatgacccagtctccatcctccctgtctgcatctgtaggagacagagtcaccatcacttg |
| | ccgggcaagtcagagcattagcagctatttaaattggtatcagcagaaaccagggaaagccccta |
| | agctcctgatctatgctgcatccagtttgcaaagtggggtcccatcaaggttcagtggcagtggatct |
| | gggacagatttcactctcaccatcagcagtctgcaacctgaagattttgcaacttactactgtcaaca |
| | gagttacagtacccctctcactttcggcggagggaccaaagtggatatcaaacgt (SEQ ID |
| | NO: 33) |

TABLE 3

| Antigen | WWT1 (Ext002 #13) | | |
|---|---|---|---|
| Peptide | RMFPNAPYL (SEQ ID NO: 1) | | |
| CDRs: | 1 | 2 | 3 |
| VH | GYSFTNFWIS | RVDPGYSYSTYSPSF | VQYSGYYDWFDP |
| | (SEQ ID NO: 34) | QG (SEQ ID NO: 35) | (SEQ ID NO: 36) |
| DNA | ggatacagcttcaccaact | agggttgatcctggctactctta | gtacaatatagtggctactatg |
| | tctggatcagc | tagcacctacagcccgtccttc | actggttcgacccc |
| | (SEQ ID NO: 37) | caaggc | (SEQ ID NO: 39) |
| | | (SEQ ID NO: 38) | |
| VL | SGSSSNIGSNTVN | SNNQRPS | AAWDDSLNGWV |
| | (SEQ ID NO: 40) | (SEQ ID NO: 41) | (SEQ ID NO: 42) |
| DNA | tctggaagcagctccaac | agtaataatcagcggccctca | gcagcatgggatgacagcct |
| | atcggaagtaatactgtaa | (SEQ ID NO: 44) | gaatggttgggtg |
| | ac (SEQ ID NO: 43) | | (SEQ ID NO: 45) |

| | |
|---|---|
| Full VH | QMQLVQSGAEVKEPGESLRISCKGSGYSFTNFWISWVRQMPGKGLE |
| | WMGRVDPGYSYSTYSPSFQGHVTISADKSTSTAYLQWNSLKASDTA |
| | MYYCARVQYSGYYDWFDPWGQGTLVTVSS (SEQ ID NO: 46) |
| DNA | cagatgcagctggtgcagtccggagcagaggtgaaagagcccggggagtctctgaggatctcct |
| | gtaagggttctggatacagcttcaccaacttctggatcagctgggtgcgccagatgcccgggaaa |
| | ggcctggagtggatggggagggttgatcctggctactcttatagcacctacagcccgtccttccaag |
| | gccacgtcaccatctcagctgacaagtctaccagcactgcctacctgcagtggaacagcctgaag |
| | gcctcggacaccgccatgtattactgtgcgagagtacaatatagtggctactatgactggttcgacc |
| | cctggggccagggaaccctggtcaccgtctcctca (SEQ ID NO: 47) |
| Full | QAVVTQPPSASGTPGQRVTISCSGSSSNIGSNTVNWYQQVPGTAPK |
| VL | LLIYSNNQRPSGVPDRFSGSKSGTSASLAISGLQSEDEADYYCAAWD |
| | DSLNGWVFGGGTKLTVLG (SEQ ID NO: 48) |
| DNA | caggctgtggtgactcagccaccctcagcgtctgggacccccgggcagagggtcaccatctcttgt |
| | tctggaagcagctccaacatcggaagtaatactgtaaactggtaccagcaggtcccaggaacgg |
| | cccccaaactcctcatctatagtaataatcagcggccctcaggggtccctgaccgattctctggctc |
| | caagtctggcacctcagcctccctggccatcagtgggctccagtctgaggatgaggctgattattac |
| | tgtgcagcatgggatgacagcctgaatggttgggtgttcggcggagggaccaagctgaccgtcct |
| | aggt (SEQ ID NO: 49) |

TABLE 4

| Antigen | WT1 (Ext002 #15) | | |
|---|---|---|---|
| Peptide | RMFPNAPYL (SEQ ID NO: 1) | | |
| CDRs | 1 | 2 | 3 |
| VH | GYNFSNKWIG | IIYPGYSDITYSPSFQG | HTALAGFDY |
| | (SEQ ID NO: 50) | (SEQ ID NO: 51) | (SEQ ID NO: 52) |
| DNA | ggctacaactttagcaaca | atcatctatcccggttactcgga | cacacagctttggccggctttg |
| | agtggatcggc | catcacctacagcccgtccttc | actac (SEQ ID NO: 55) |
| | (SEQ ID NO: 53) | caaggc (SEQ ID NO: 54) | |
| VL | RASQNINKWLA | KASSLES | QQYNSYAT |
| | (SEQ ID NO: 56) | (SEQ ID NO: 57) | (SEQ ID NO: 58) |
| DNA | Cgggccagtcagaatatc | aaggcgtctagtttagaaagt | caacaatataatagttatgcga |
| | aataagtggctggcc | (SEQ ID NO: 60) | cg (SEQ ID NO: 61) |
| | (SEQ ID NO: 59) | | |

| | |
|---|---|
| Full | QVQLVQSGAEVKKPGESLKISCKGSGYNFSNKWIGWVRQLPGRGLE |
| VH | WIAIIYPGYSDITYSPSFQGRVTISADTSINTAYLHWHSLKASDTAMYYC |
| | VRHTALAGFDYWGLGTLVTVSS (SEQ ID NO: 62) |
| DNA | caggtgcagctggtgcagtctggagcagaggtgaaaaagcccggagagtctctgaagatctcctg |
| | taagggttctggctacaactttagcaacaagtggatcggctgggtgcgccaattgcccgggagagg |
| | cctggagtggatagcaatcatctatcccggttactcggacatcacctacagcccgtccttccaaggc |
| | cgcgtcaccatctccgccgacacgtccattaacaccgcctacctgcactggcacagcctgaaggc |
| | ctcggacaccgccatgtattattgtgtgcgacacacagctttggccggctttgactactggggcctgg |
| | gcaccctggtcaccgtctcctca |
| | (SEQ ID NO: 63) |
| Full | DIQMTQSPSTLSASVGDRVTITCRASQNINKWLAWYQQRPGKAPQLLI |
| VL | YKASSLESGVPSRFSGSGSGTEYTLTISSLQPDDFATYYCQQYNSYAT |
| | FGQGTKVEIKR (SEQ ID NO: 64) |
| DNA | gacatccagatgacccagtctccttccaccctgtctgcatctgtaggagacagagtcacaatcacttg |
| | ccgggccagtcagaatatcaataagtggctggcctggtatcagcagagaccagggaaagcccct |
| | cagctcctgatctataaggcgtctagtttagaaagtggggtcccatctaggttcagcggcagtggatc |
| | tgggacagaatacactctcaccatcagcagcctgcagcctgatgattttgcaacttattactgccaac |
| | aatataatagttatgcgacgttcggccaagggaccaaggtggaaatcaaacgt (SEQ ID NO: |
| | 65) |

TABLE 5

| Antigen | WT1 (Ext002 #18) | | |
|---|---|---|---|
| Peptide | RMFPNAPYL (SEQ ID NO: 1) | | |
| CDRs: | 1 | 2 | 3 |
| VH | GFTFDDYGMS | GINWNGGSTGYADS | ERGYGYHDPHDY |
| | (SEQ ID NO: 66) | VRG (SEQ ID NO: 67) | (SEQ ID NO: 68) |
| DNA | gggttcacctttgatgattat | ggtattaattggaatggtggt | gagcgtggctacgggtacca |
| | ggcatgagc | agcacaggttatgcagactc | tgatccccatgactac |
| | (SEQ ID NO: 69) | tgtgaggggc (SEQ ID | (SEQ ID NO: 71) |
| | | NO: 70) | |
| VL | GRNNIGSKSVH | DDSDRPS | QVWDSSSDHVV |
| | (SEQ ID NO: 72) | (SEQ ID NO: 73) | (SEQ ID NO: 74) |
| DNA | gggagaaacaacattgg | gatgatagcgaccggccctc | caggtgtgggatagtagtagt |
| | aagtaaaagtgtgcac | a | gatcatgtggta |
| | (SEQ ID NO: 75) | (SEQ ID NO: 76) | (SEQ ID NO: 77) |

| | |
|---|---|
| Full | EVQLVQSGGGVVRPGGSLRLSCAASGFTFDDYGMSWVRQAPGKG |
| VH | LEWVSGINWNGGSTGYADSVRGRFTISRDNAKNSLYLQMNSLRAE |
| | DTALYYCARERGYGYHDPHDYWGQGTLVTVSS (SEQ ID NO: 78) |
| DNA | gaagtgcagctggtgcagtctgggggaggtgtggtacggcctggggggtccctgagactctcct |
| | gtgcagcctctgggttcacctttgatgattatggcatgagctgggtccgccaagctccagggaag |
| | gggctggagtgggtctctggtattaattggaatggtggtagcacaggttatgcagactctgtgagg |
| | ggccgattcaccatctccagagacaacgccaagaactccctgtatctgcaaatgaacagtctg |
| | agagccgaggacacggccttgtattactgtgcgagagagcgtggctacgggtaccatgatccc |
| | catgactactggggccaaggcaccctggtgaccgtctcctca (SEQ ID NO: 79) |
| Full | QSVVTQPPSVSVAPGKTARITCGRNNIGSKSVHWYQQKPGQAPVL |
| VL | VVYDDSDRPSGIPERFSGSNSGNTATLTISRVEAGDEADYYCQVW |
| | DSSSDHVVFGGGTKLTVLG (SEQ ID NO: 80) |
| DNA | cagtctgtcgtgacgcagccgccctcggtgtcagtggccccaggaaagacggccaggattac |
| | ctgtgggagaaacaacattggaagtaaaagtgtgcactggtaccagcagaagccaggccag |
| | gcccctgtgctggtcgtctatgatgatagcgaccggccctcagggatccctgagcgattctctgg |
| | ctccaactctgggaacacggccaccctgaccatcagcagggtcgaagccggggatgaggcc |
| | gactattactgtcaggtgtgggatagtagtagtgatcatgtggtattcggcggagggaccaagct |
| | gaccgtcctaggt (SEQ ID NO: 81) |

TABLE 6

| Antigen | W1 (Ext002 #23) | | |
|---|---|---|---|
| Peptide | RMFPNAPYL (SEQ ID NO. 1) | | |
| CDRs | 1 | 2 | 3 |
| VH | GFSVSGTYMG | LLYSGGGTYHPASLQ | GGAGGGHFDS |
| | (SEQ ID NO. 82) | G | (SEQ ID NO. 84) |
| | | (SEQ ID NO. 83) | |
| DNA | gggttctccgtcagtggcac | cttctttatagtggtggcggcac | gaggggcaggaggtggcc |
| | ctacatgggc (SEQ ID | ataccacccagcgtccctgca | actttgactcc (SEQ ID |
| | NO. 85) | gggc | NO. 87) |
| | | (SEQ ID NO. 86) | |
| VL | TGSSSNIGAGYDVH | GNSNRPS | AAWDDSLNGYV |
| | (SEQ ID NO. 88) | (SEQ ID NO. 89) | (SEQ ID NO. 90) |
| DNA | actgggagcagctccaac | ggtaacagcaatcggccctca | gcagcatgggatgacagcct |
| | atcggggcaggttatgatgt | (SEQ ID NO. 92) | gaatggttatgtc |
| | acac | | (SEQ ID NO. 93) |
| | (SEQ ID NO. 91) | | |

| | |
|---|---|
| Full | EVQLVETGGGLLQPGGSLRLSCAASGFSVSGTYMGWVRQAPGKGLE |
| VH | WVALLYSGGGTYHPASLQGRFIVSRDSSKNMVYLQMNSLKAEDTAVY |
| | YCAKGGAGGGHFDSWGQGTLVTVSS (SEQ ID NO. 94) |
| DNA | gaggtgcagctggtggagaccggaggaggcttgctccagccgggggggtccctcagactctcctg |
| | tgcagcctctgggttctccgtcagtggcacctacatgggctgggtccgccaggctccagggaaggg |
| | actggagtgggtcgcacttctttatagtggtggcggcacataccacccagcgtccctgcagggccg |
| | attcatcgtctccagagacagctccaagaatatggtctatcttcaaatgaatagcctgaaagccgag |
| | gacacggccgtctattactgtgcgaaaggaggggcaggaggtggccactttgactcctggggcca |
| | aggcaccctggtgaccgtctcctca |
| | (SEQ ID NO. 95) |
| Full | QSVLTQPPSVSGAPGQRVTISCTGSSSNIGAGYDVHWYQQLPGTAPK |
| VL | LLIYGNSNRPSGVPDRFSGSKSGTSASLAISGLQSEDEADYYCAAWD |
| | DSLNGYVFGTGTKLTVLG (SEQ ID NO. 96) |
| DNA | cagtctgtgttgacgcagccgccctcagtgtctggggccccagggcagagggtcaccatctcctgc |
| | actgggagcagctccaacatcggggcaggttatgatgtacactggtaccagcagcttccaggaac |
| | agcccccaaactcctcatctatggtaacagcaatcggccctcaggggtccctgaccgattctctggc |
| | tccaagtctggcacctcagcctccctggccatcagtgggctccagtctgaggatgaggctgattatta |
| | ctgtgcagcatgggatgacagcctgaatggttatgtcttcggaactgggaccaagctgaccgtccta |
| | ggt (SEQ ID NO. 97) |

In constructing a recombinant immunoglobulin containing the desired antigen-binding region, the sequences shown above can be used in combination with appropriate amino acid sequences for constant regions of various immunoglobulin isotypes using methods for the production of a wide array of antibodies that are known to those of skill in the art. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. In one aspect, the light or heavy chain constant region is a fragment, derivative, variant, or mutein of a naturally occurring constant region. In one embodiment, however, light and heavy chain constant regions may have the amino acid sequences (as shown in Table 7) of SEQ ID NO. 98 and SEQ ID NO. 99, respectively.

TABLE 7

| | |
|---|---|
| LC constant | QPKANPTVTLFPPSSEELQANKATLVCLISDFYPGAVT |
| region | VAWKADGSPVKAGVETTKPSKQSNNKYAASSYLSLTPE |
| | QWKSHRSYSCQVTHEGSTVEKTVAPTECS |
| | (SEQ ID NO: 98) |
| HC constant | TKGPSVFPLAPSSKSTSGGTAALGCLVKDYFPEPVTVS |
| region | WNSGALTSGVHTFPAVLQSSGLYSLSSVVTVPSSSLGT |
| | QTYICNVNHKPSNTKVDKRVEPKSCDKTHTCPPCPAPE |
| | LLGGPSVFLFPPKPKDTLMISRTPEVTCVVVDVSHEDP |
| | EVKFNWYVDGVEVHNAKTKPREEQYNSTYRVVSVLTVL |
| | HQDWLNGKEYKCKVSNKALPAPIEKTISKAKGQPREPQ |
| | VYTLPPSRDELTKNQVSLTCLVKGFYPSDIAVEWESNG |
| | QPENNYKTTPPVLDSDGSFFLYSKLTVDKSRWQQGNVF |
| | SCSVMHEALHNHYTQKSLSLSPGK |
| | (SEQ ID NO: 99) |

In one embodiment, the antibody of the invention comprises a light chain and heavy chain with amino acid sequences as follows:

TABLE 8

| | |
|---|---|
| Complete light | 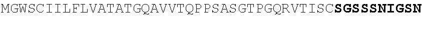 |
| chain | TVNVVYQQVPGTAPKLLIYSNNQRPSGVPDRFSGSKSGTSASLAISGL |
| | QSEDEADYYCAAWDDSLNGWVFGGGTKLTVLGQPKANPTVTLFPPS |
| | SEELQANKATLVCLISDFYPGAVTVAWKADGSPVKAGVETTKPSKQS |
| | NNKYAASSYLSLTPEQWKSHRSYSCQVTHEGSTVEKTVAPTECS |
| | (SEQ ID NO: 100) |
| Complete | 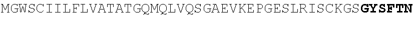 |
| heavy chain | FWISVVVRQMPGKGLEWMGRVDPGYSYSTYSPSFQGHVTISADKSTS |
| | TAYLQWNSLKASDTAMYYCARVQYSGYYDWFDPWGQGTLVTVSSA |
| | STKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDK |
| | RVEPKSCDKTHTCPPCPAPELLGGPSVFLFPPKPKDTLMISRTPEVTC |
| | VVVDVSHEDPEVKFNVVYVDGVEVHNAKTKPREEQYNSTYRVVSVLT |
| | VLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK* |
| | (SEQ ID NO: 101) |

A leader sequence, MGWSCIILFLVATATG (SEQ ID NO: 102), as shown in gray may be included. CDRs are bolded in Table 8 and correspond to the CDRs listed in Table 3.

In each of Tables 1-6, a nucleic acid that encodes for the variable and hypervariable (CDR) regions of the heavy or light chain is also shown. Vectors and other nucleic acid constructs which comprise a nucleic acid that encodes the antibody and which can be used for expression of the antibodies from MAGE 1.5 CHO cells are also encompassed by the invention. The antibodies of the present disclosure also include substantially homologous polypeptides having antigen-binding portions that are at least 70%, at least 75%, at least 80%, at least 85%, at least 90%, at least 95%, at least 97%, at least 98%, or at least 99% identical to the peptides described in Tables 1-6 or 8. In one aspect, an antibody of the present disclosure comprises a heavy chain variable region comprising CDR1, CDR2, and CDR3 from a VH sequence in any of Tables 1-6 or 8 that is at least 90% identical to that VH sequence and/or comprises a light chain variable region comprising CDR1, CDR2, and CDR3 from a VL sequence in Tables 1-6 or 8 that is at least 90% identical to that VL sequence. For example, in one aspect, an antibody according to the present disclosure comprises a heavy chain variable region comprising CDR1, CDR2, and CDR3 from SEQ ID NO: 101 that is at least 90%, at least 95%, at least 97%, at least 98%, or at least 99% identical to SEQ ID NO: 101 and/or comprises a light chain variable region comprising CDR1, CDR2, and CDR3 from SEQ ID NO: 100 that is at least 90%, at least 95%, at least 97%, at least 98%, or at least 99% identical to SEQ ID NO: 100.

In one aspect, the present disclosure provides an antibody comprising: (A) a heavy chain (HC) variable region comprising HC-CDR1, HC-CDR2 and HC-CDR3 respectively, comprising amino acid sequences SEQ ID NOS: 2, 3, and 4; 18, 19 and 20; 34, 35, and 36; 50, 51, and 52; 66, 67, and 68 or 82, 83, and 84; and a light chain (LC) variable region comprising LC-CDR1, LC-CDR2 and LC-CDR3 respectively, comprising amino acid sequences SEQ ID NOS: 8, 9 and 10; 24, 25 and 26; 40, 41 and 42; 56, 57 and 58; 72, 73 and 74 or 88, 89 and 90; or (B) a VH and VL comprising the amino acid sequence of SEQ ID NO: 14 and SEQ ID NO: 16; 30 and 32; 46 and 48; 62 and 64; 78 and 80 or 94 and 96, respectively, wherein said antibody has no detectable fucose or galactose.

In one aspect, the antibody comprises a heavy chain (HC) variable region comprising HC-CDR1, HC-CDR2 and HC-CDR3 respectively, comprising amino acid sequences SEQ ID NOS: 2, 3, and 4; and a light chain (LC) variable region comprising LC-CDR1, LC-CDR2 and LC-CDR3 respectively, comprising amino acid sequences SEQ ID NOS: 8, 9 and 10.

In another aspect, the antibody comprises a heavy chain (HC) variable region comprising HC-CDR1, HC-CDR2 and HC-CDR3 respectively, comprising amino acid sequences SEQ ID NOS: 18, 19 and 20; and a light chain (LC) variable region comprising LC-CDR1, LC-CDR2 and LC-CDR3 respectively, comprising amino acid sequences SEQ ID NOS: 24, 25 and 26.

In another aspect, the antibody comprises a heavy chain (HC) variable region comprising HC-CDR1, HC-CDR2 and HC-CDR3 respectively, comprising amino acid sequences SEQ ID NOS: 34, 35, and 36; and a light chain (LC) variable region comprising LC-CDR1, LC-CDR2 and LC-CDR3 respectively, comprising amino acid sequences SEQ ID NOS: 40, 41 and 42.

In another aspect, the antibody comprises a heavy chain (HC) variable region comprising HC-CDR1, HC-CDR2 and HC-CDR3 respectively, comprising amino acid sequences SEQ ID NOS: 50, 51, and 52; and a light chain (LC) variable region comprising LC-CDR1, LC-CDR2 and LC-CDR3 respectively, comprising amino acid sequences SEQ ID NOS: 56, 57 and 58.

In another aspect, the antibody comprises a heavy chain (HC) variable region comprising HC-CDR1, HC-CDR2 and HC-CDR3 respectively, comprising amino acid sequences SEQ ID NOS: 66, 67, and 68; and a light chain (LC) variable region comprising LC-CDR1, LC-CDR2 and LC-CDR3 respectively, comprising amino acid sequences SEQ ID NOS: 72, 73 and 74.

In another aspect, the antibody comprises a heavy chain (HC) variable region comprising HC-CDR1, HC-CDR2 and HC-CDR3 respectively, comprising amino acid sequences SEQ ID NOS: 82, 83, and 84; and a light chain (LC) variable region comprising LC-CDR1, LC-CDR2 and LC-CDR3 respectively, comprising amino acid sequences SEQ ID NOS: 88, 89 and 90.

In one embodiment, the antibody comprises a light chain consisting essentially of the amino acid sequence of SEQ ID NO: 100 and a heavy chain consisting essentially of the amino acid sequence of SEQ ID NO: 101.

In one aspect, an antibody of the present disclosure specifically binds to WT-1 peptide RMFPNAPYL (SEQ ID NO: 1) in conjunction with HLA/A2. Optionally, the HLA-A2 is HLA-A0201. In another aspect, the antibody exhibits between 50-100% (80%) higher affinity for activating human FcγRIIIa (158V variant) than normally glycosylated antibody, has 3- to 4-fold (3.5-fold) higher affinity for a FcγRIIIa 158F variant than normally glycosylated antibody, and has between 30 and 70% (50%) reduced affinity for inhibitory FcγRIIb than normally glycosylated antibody.

In other aspects, the present disclosure provides an isolated nucleic acid that encodes an antibody described herein, a vector comprising said nucleic acid, and a cell comprising said nucleic acid or said vector. In another aspect, the present disclosure provides a kit comprising an antibody described herein.

In another aspect, the invention relates to a derivative or analog of an antibody of the present disclosure. A derivative can comprise any molecule or substance that imparts a desired property, such as increased half-life in a particular use. Examples of molecules that can be used to form a derivative include, but are not limited to, albumin (e.g., human serum albumin) and polyethylene glycol (PEG). Derivatives such as albumin-linked and PEGylated derivatives of antibodies can be prepared using techniques well known in the art. An analog may be a non-peptide analog of an antibody described herein. Non-peptide analogs are commonly used in the pharmaceutical industry as drugs with properties analogous to those of the template peptide. These types of non-peptide compound are termed “peptide mimetics” or “peptidomimetics,” (Fauchere, J. Adv. Drug Res 1986; 15:29; Veber and Freidinger TINS 1985; p. 392; Evans et al. J. Med. Chem 1987; 30:1229). Peptide mimetics that are structurally similar to the antibodies of the present disclosure may be used to produce an equivalent therapeutic or prophylactic effect. Generally, peptidomimetics are structurally similar to a paradigm polypeptide (i.e., a polypeptide that has a desired biochemical property or pharmacological activity), such as a human antibody, but have one or more peptide linkages optionally replaced by a linkage selected from the group consisting of: —CH₂NH—, —CH₂S—, —CH₂—CH₂—, —CH═CH-(cis and trans), —COCH₂—, —CH(OH)CH₂—, and —CH₂SO—, by methods well known in the art.

Methods for the recovery and purification of antibodies are well known in the art. Antibodies according to the present disclosure may be prepared by any of a number of conventional techniques. For example, they may be produced in recombinant expression systems, using any technique known in the art. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988). Certain of the techniques involve isolating a nucleic acid encoding a polypeptide chain (or portion thereof) of an antibody of interest, and manipulating the nucleic acid through recombinant DNA technology. The nucleic acid may be fused to another nucleic acid of interest, or altered (e.g., by mutagenesis or other conventional techniques) to add, delete, or substitute one or more amino acid residues.

Any expression system known in the art can be used to make the recombinant antibodies of the present disclosure. In general, host cells are transformed with a recombinant expression vector that comprises DNA encoding a desired polypeptide. Among the host cells that may be employed are prokaryotes, yeast or higher eukaryotic cells. Prokaryotes include gram negative or gram positive organisms, for example E. coli or bacilli. Higher eukaryotic cells include insect cells and established cell lines of mammalian origin. Examples of suitable mammalian host cell lines include the COS-7 line of monkey kidney cells (ATCC CRL 1651) (Gluzman et al., Cell 1981; 23:175), L cells, 293 cells, C127 cells, 3T3 cells (ATCC CCL 163), Chinese hamster ovary (CHO) cells, HeLa cells, BHK (ATCC CRL 10) cell lines, and the CVI/EBNA cell line derived from the African green monkey kidney cell line CVI (ATCC CCL 70) as described by McMahan et al., EMBO J 1991; 10: 2821. Appropriate cloning and expression vectors for use with bacterial, fungal, yeast, and mammalian cellular hosts are described in the art, e.g., by Pouwels et al., Cloning Vectors: A Laboratory Manual, Elsevier, N.Y., 1985.

The transformed cells can be cultured under conditions that promote expression of the polypeptide, and the polypeptide recovered by conventional protein purification procedures. One such purification procedure includes the use of affinity chromatography, e.g., over a matrix having all or a portion of the antigen bound thereto. Polypeptides contemplated for use herein include substantially homogeneous recombinant antibodies substantially free of contaminating endogenous materials.

The resulting antibody has an amino acid sequence as described above and no detectable fucose or galactose as part of the carbohydrate of the antibody. For example, the fucose content and/or galactose content of the antibody can be reduced by at least 30%, at least 40%, at least 50%, at least 60%, at least 70%, at least 80%, at least 90%, or 100% compared to the wildtype antibody.

In another aspect, the present disclosure provides a pharmaceutical composition comprising an antibody described herein and a physiologically acceptable diluent, excipient, or carrier. In one aspect, a pharmaceutical composition of the present disclosure comprises a antibody described herein with one or more substances selected from the group consisting of a buffer, an antioxidant such as ascorbic acid, a low molecular weight polypeptide (such as those having fewer than 10 amino acids), a protein, an amino acid, a carbohydrate, a chelating agent such as EDTA, glutathione, a stabilizer, and an excipient. Neutral buffered saline or saline mixed with serum albumin are examples of appropriate diluents. In accordance with appropriate industry standards, preservatives such as benzyl alcohol may also be added. A liquid pharmaceutical composition may include, for example, one or more of the following: a sterile diluent such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition is preferably sterile. In one aspect, the composition may be formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in Remington's Pharmaceutical Sciences, 16th Ed. (1980) and 20th Ed. (2000), Mack Publishing Company, Easton, Pa.

As is understood in the art, pharmaceutical compositions comprising the antibodies of the present disclosure are administered to a subject in a manner appropriate to the indication. A pharmaceutical composition of the present disclosure comprising an antibody described herein may be formulated for delivery by any route that provides an effective dose of the antibody. Pharmaceutical compositions may be administered by any suitable technique, including but not limited to, parenterally, topically, or by inhalation. If injected, the pharmaceutical composition can be administered, for example, via intra-articular, intravenous, intramuscular, intralesional, intraperitoneal or subcutaneous routes, by bolus injection, or continuous infusion. Localized administration, e.g., at a tumor site, is contemplated, as are transdermal delivery and sustained release from implants. Delivery by inhalation includes, for example, nasal or oral inhalation, use of a nebulizer, inhalation of the antagonist in aerosol form, and the like. Other alternatives include eyedrops; oral preparations including tablets, capsules, syrups, lozenges or chewing gum; and topical preparations such as lotions, gels, sprays, patches, and ointments.

In one aspect, the present disclosure provides use of an antibody described herein, e.g., in the preparation of a medicament, for the treatment of a WT1 positive disease. In another aspect, the present disclosure provides a method for treatment of a subject having a WT1-positive disease, comprising administering to the subject a therapeutically effective amount of an antibody or antigen binding fragment described herein. In one aspect, the WT1-positive disease is a chronic leukemia or acute leukemia or a WT1+ cancer, for example, a WT1-positive disease selected from the group consisting of chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma

The methods of treatment and uses of the present disclosure encompass alleviation or prevention of at least one symptom or other aspect of a disorder, or reduction of disease severity, and the like. In one aspect, a therapeutically effective amount of an antibody or pharmaceutical composition of the invention is an amount effective to inhibit growth of WT1-positive cells, reduce tumor size/burden, prevent tumor cell metastasis/infiltration, and/or result in cell death, e.g., via apoptosis or necrosis. An antibody or pharmaceutical composition described herein need not effect a complete cure, or eradicate every symptom or manifestation of a disease, to constitute a viable therapeutic agent. As is recognized in the art, therapeutic agents may reduce the severity of a given disease state, but need not abolish every manifestation of the disease to be regarded as useful. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient.

Dosages and the frequency of administration for use in the methods of the present disclosure may vary according to such factors as the route of administration, the particular antibodies employed, the nature and severity of the disease to be treated, whether the condition is acute or chronic, and the size and general condition of the subject. Appropriate dosages can be determined by procedures known in the pertinent art, e.g., in clinical trials that may involve dose escalation studies.

An antibody of the present disclosure may be administered, for example, once or more than once, e.g., at regular intervals over a period of time. In general, the antibody or pharmaceutical composition is administered to a subject until the subject manifests a medically relevant degree of improvement over baseline for the chosen indicator or indicators.

In general, the amount of an antibody described herein present in a dose, or produced in situ by an encoding polynucleotide present in a dose, ranges from about 10 μg per kg to about 20 mg per kg of host. The use of the minimum dosage that is sufficient to provide effective therapy is usually preferred. Patients may generally be monitored for therapeutic or prophylactic effectiveness using assays suitable for the condition being treated or prevented; assays will be familiar to those having ordinary skill in the art and some are described herein.

The methods disclosed herein may include oral administration of an antibody described herein or delivery by injection of a liquid pharmaceutical composition. When administered in a liquid form, suitable dose sizes will vary with the size of the subject, but will typically range from about 1 ml to about 500 ml (comprising from about 0.01 μg to about 1000 μg per kg) for a 10 kg to 60 kg subject. Optimal doses may generally be determined using experimental models and/or clinical trials. The optimal dose may depend upon the body mass, body area, weight, or blood volume of the subject. As described herein, the appropriate dose may also depend upon the patient's condition, that is, stage of the disease, general health status, age, gender, weight, and other factors familiar to a person skilled in the medical art.

In particular embodiments of the methods and uses described herein, the subject is a human or non-human animal. A subject in need of the treatments described herein may exhibit symptoms or sequelae of a disease, disorder, or condition described herein or may be at risk of developing the disease, disorder, or condition. Non-human animals that may be treated include mammals, for example, non-human primates (e.g., monkey, chimpanzee, gorilla), rodents (e.g., rats, mice, gerbils, hamsters, ferrets, rabbits), lagomorphs, swine (e.g., pig, miniature pig), equine, canine, feline, bovine, and other domestic farm and zoo animals.

The present disclosure will be more readily understood by reference to the following Examples, which are provided by way of illustration and are not intended to be limiting.

### Examples

### Oligosaccharide Analysis and FcR Binding Assays

N-Glycan from ESK1 or ESKM antibodies was cleaved from antibody by PNGase F, and measured by HPAEC-PAD using PA200 column. Binding of ESK1/ESKM antibodies to mouse FcγR4 and mouse FcγRIIb were measured by ELISA. Briefly, 2 μg/mL recombinant mouse FcγR4 or FcγRIIb were coated onto ELISA plate. Various concentrations of ESK1 or ESKM antibodies were added to the wells for 1 hour at room temperature, then detected by secondary antibody (HRP conjugated anti-human IgG Fab′2 fragment). Binding of ESK1/ESKM to human FcγRs was measured by Flow Cytometry (Guava easyCyte HT, Millipore) against CHO cells expressing appropriate human FcγR. Binding of ESK1/ESKM to human FcγRI, FcγRIIa, FcγRIIIa-158V, FcγRIIIa-158F and human FcRn were measured directly using ESK1 or ESKM antibody, followed by the 2^{nd} antibody (FITC conjugated Fab′2 fragment anti-human IgG Fab′2). For human FcγRIIb, dimers were formed first by mixing ESK1 or ESKM to a PE-conjugated Fab′2 fragment anti-human Fab′2 at 2:1 ratio at RT for 2 hour. Binding of dimeric complex of ESK1 or ESKM to human FcγRIIb were measured directly by Flow Cytometry using the immunocomplex.

In another assay, ESK1 was expressed in Chinese Hamster Ovary cells using the GlymaxX® technology (ProBioGen, Berlin, Germany) to reduce the fucose content of the antibody. ESKM antibody was purified from two separate pools of cells, and fucose reduction confirmed by mass spectrometry to be 70% reduced or 100% reduced. Both the ESKM having 70% reduction in fucosylation and the ESKM having 100% reduction in fucosylation (completely a-fucosylated) batches were compared to ESK1 as a wildtype IgG1 and to ESK1 containing D265A/P329A mutations in the Fc domain (ESK1 DAPA) that eliminated binding to human FcgRIIIa in ADCC killing assays. T2 cells were pulsed with 25 mg/ml RMFPNAPYL (SEQ ID NO:1) peptide overnight. The next day, 15,000 pulsed cells were added to serially diluted ESK-1 antibodies. Then 90,000 Jurkat cells transduced to express CD16A and an NFAT-luciferase reporter were added. Plates were gently mixed and spun at 200×g for 4 minutes and then incubated at 37° C. for 4 hours. At the end of the incubation, the plates were brought to room temp for ˜15 minutes, 60 μl of Brightlite™ (Perkin Elmer) was then added to each well. Plates were shaken for 3 minutes and analyzed on the EnVision Multilabel Reader (PerkinElmer).

### Cell Lines and Reagents

Cell lines were from laboratory stocks, and were maintained in RPMI with 10% FBS. Peptides for T2 pulsing assays were purchased and synthesized by Genemed Synthesis, Inc. (San Antonio, Tex.). Peptides were >90% pure. GFP+ luciferase-expressing SET2 and JMN cells were generated as described previously (Dao T et al. Science translational medicine 2013; 5(176):176ra33). All cells were HLA typed.

### Animals

C57BL/6 and C57BL/-Tg (HLA-A2.1) 1 Enge/J (6-8 week-old male), and NOD.Cg-Prkdc^{scid}//2rg^{tm1Wjl}/SzJ mice (6-8 week-old male), known as NOD scid gamma (NSG), were purchased from Jackson Laboratory (Bar Harbor, Me.). NOD.Cg-Prkdc^{scid}//2rg^{tm1Sug}/JicTac (NOG), and C.B-Igh-1^{b}/IcrTac-Prkdc_{scid} (SCID) were purchased from Taconic (Hudson, N.Y.). All studies were conducted in accordance with IACUC approved protocols.

### Antibody-Dependent Cellular Cytotoxicity (ADCC)

Peripheral blood mononuclear cells (PBMCs) from healthy donors were obtained by Ficoll density centrifugation. Target cells used for ADCC were T2 cells pulsed with or without WT1 or RHAMM-3 peptides, and cancer cell lines without peptide pulsing. ESK1, ESKM or isotype control human IgG1 at various concentrations were incubated with target cells and fresh PBMCs at different effector: target (E:T) ratio. Cytotoxicity was measured by standard 4 hour ⁵¹Cr-release assay.

In another assay, 5×10⁶ OV56 human ovarian cancer cells were collected, washed and resuspended in CalceinAM. After a 50-minute incubation, cells were washed twice with PBS and added to the assay plate containing serially diluted antibodies. Purified human NK cells from leukopak (HemaCare, Van Nuys, Calif.) were added in an effector to target ratio of 20:1. Cells were incubated at 37° C. for 3.5 hours and Calcein release was measured on EnVision Multilabel Reader (PerkinElmer, Waltham, Mass.). Specific lysis was calculated as (sample−spontaneous release)/(max release−spontaneous release)*100%.

### Therapy of ESK1 and ESKM in Human Mesothelioma, AML and ALL Xenograft Mouse Models

Luciferase-expressing JMN cells (3×10⁵) were injected into the intraperitoneal cavity of CB17 SCID mice. On day 4, tumor engraftment was confirmed by luciferase imaging, signal was quantified with Living Image® software (Xenogen), and mice were sorted into groups with similar average signal from the supine position. Mice were injected intraperitoneally with 50 μg ESK1, ESKM or human isotype IgG1 antibody twice weekly beginning on day 4.

For AML leukemia studies, luciferase-expressing BV173 (Ph+ ALL) or SET2 (AML) cells (3×10⁶) were injected intravenously via tail vein into NSG mice. Animals were sorted, and, where indicated, treated with intraperitoneal injections of 100 μg ESKM twice weekly beginning on day 6.

For ALL leukemia studies, fresh pre-B cell ALL cells were obtained under IRB approved protocols from the CNS relapse of a female pediatric patient after treatment with a chemotherapy induction regimen and bone-marrow transplant. Leukemia cells were transduced with a lentiviral vector containing a plasmid encoding luciferase/GFP. Luciferase+/GFP+ leukemia was then expanded in NSG mice, luciferase signal was confirmed by bioluminescent imaging, and tumor cells were harvested and sorted for CD45. Leukemia cells (5.5×10⁶/animal) were then injected intravenously into NSG mice, and engraftment was confirmed by bioluminescent imaging on day 2 post-injection. Animals were sorted into two groups (n=5 each) so that average signal in each group was equal. ESKM or isotype control antibody (100 μg/animal) was administered via retro-orbital injection on days 2, 5, 9, 12, 14 and 23, and leukemia growth was followed by bioluminescent imaging. On day 41, animals were sacrificed and bone marrow cells were harvested and pooled: after dissection and homogenization, cells were centrifuged, subjected to Ficoll density centrifugation, and counted after red blood cell lysis (acetic acid). An equal number of cells from each treatment group was resuspended in matrigel (200 μL/injection) and engrafted SC into the opposite shoulders of NSG mice (n=4). No further treatment was given, and tumor growth was followed by bioluminescent imaging.

### Pharmacokinetic and Biodistribution Studies

Antibody was labeled with ¹²⁵I (PerkinElmer) using the chloramine-T method. 100 μg antibody was reacted with 1 mCi ¹²⁵I and 20 μg chloramine-T, quenched with 200 μg Na metabisulfite, then separated from free ¹²⁵I using a 10DG column equilibrated with 2% bovine serum albumin in PBS. Specific activities of products were in the range of 4-8 mCi/mg. Radiolabeled mAb was injected into mice retro-orbitally, and blood and/or organs were collected at various time points, weighed and measured on a gamma counter.

### Toxicity Studies

For isolated cell binding studies, C57BL6/J or HLA-A2.1+ transgenic mice were sacrificed, and cells were harvested from spleen, thymus and bone-marrow. After red blood cell lysis, cells (10⁶ per tube, in duplicate) were incubated with ¹²⁵I-labeled ESK1 (1 μg/ml) for 45 minutes on ice, then washed extensively with 1% bovine serum albumin in PBS on ice. To determine specific binding, a set of cells was assayed after pre-incubation in the presence of 50-fold excess unlabeled ESK1 for 20 minutes on ice. Bound radioactivity was measured by a gamma counter, specific binding was determined, and the number of bound antibodies per cell was calculated from specific activity.

For toxicity studies, 100 μg of ESKM or isotype control mAb was injected into human HLA-A0201 transgenic mice (Jackson Labs) on days 0 and 4, to mimic the maximum dose and therapeutic schedule used in the therapy experiments. Mice were sacrificed on day 5 for collection and analysis of whole blood and bone marrow leukocytes. Whole blood was analyzed with a Hemavet system (Drew Scientific). Bone marrow cells were harvested from both femurs and tibias of mice and subjected to red blood cell lysis, then analyzed by flow cytometry (see Antibodies and flow cytometry analysis).

Alternatively, mice treated as above were sacrificed on day 6 for histolo-pathologic examination of major organs and possible WT1 positive target organs (spleen, bone and bone marrow, liver, thymus, kidney) as well as heart, lung, and ileum. Mice were sacrificed and whole organs were collected, fixed (4% paraformaldehyde), decalcified in EDTA where necessary (femurs only), embedded in paraffin, sectioned and stained with H&E.

### Antibodies and Flow Cytometry Analysis

For cell surface staining, cells were incubated with appropriate mAbs for 30-60 minutes on ice, washed, and incubated with secondary antibody reagents when necessary. Flow cytometry data were collected on a FACS Calibur or LSRFortessa (Becton Dickinson) and analyzed with FlowJo software. APC-labeled ESK1 and hIgG1 isotype antibodies were generated with Lightning-Link® kit (Innova Biosciences).

For HSC toxicity studies, mouse bone marrow cells were stained with the following antibodies: (Lineage; CD3, CD4, CD8, Gr1, B220, CD19, TER119, all conjugated with PE-Cy5), Sca-Pacific Blue, CD34-FITC, SLAM-APC, CD48-PE and c-KIT-AlexaFluor 780. The stained cells were analyzed for flow cytometry on the BD LSRII instrument.

For mouse immunophenotyping, cells were isolated from the intraperitoneal cavity by washing with complete media, or from spleen by dissection and red blood cell lysis (RBC Lysis Solution, Qiagen). Samples were then analyzed by flow cytometry after multi-color staining with well-characterized lineage-specific markers: CD335 (NKp46)-PE and F4/80 (BM8)-AlexaFluor700 (BioLegend), CD49b/VLA-2a (DX5)-FITC (Life Technologies), CD3e-PE-Cy7 and Gr-1/Ly-6G/Ly-6C (RB6-8C5)-PerCP-Cy5.5 (BD Pharmingen).

### ESKM Antibody has Enhanced Binding Affinity for FcγRIIIa and Reduced Affinity for FcγRIIb

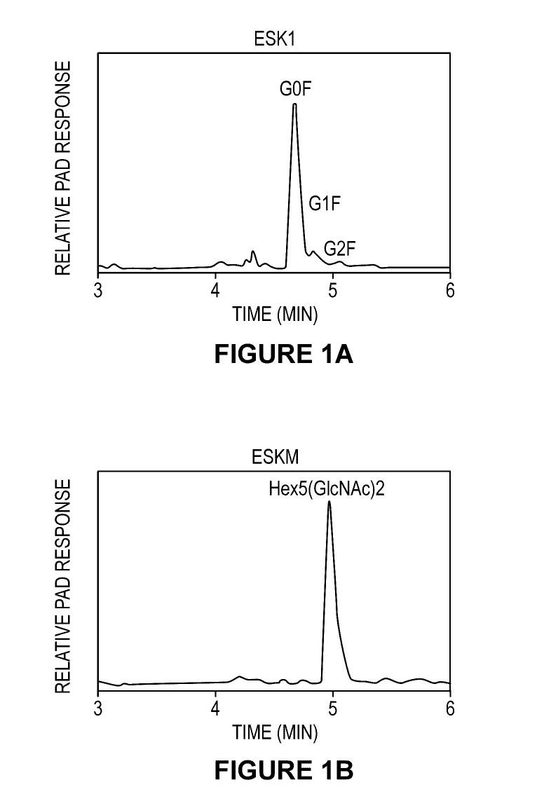

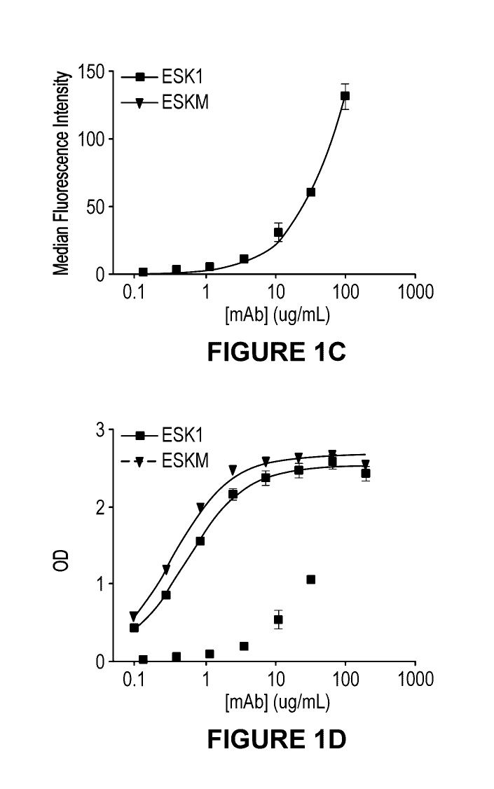

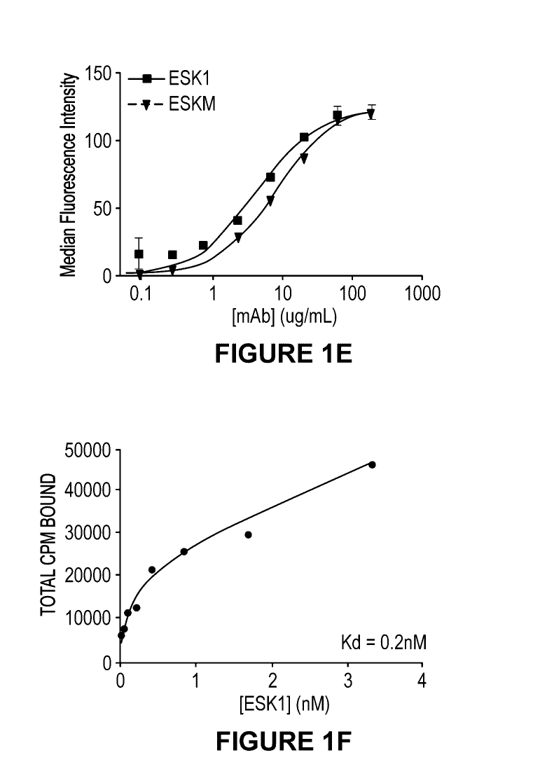

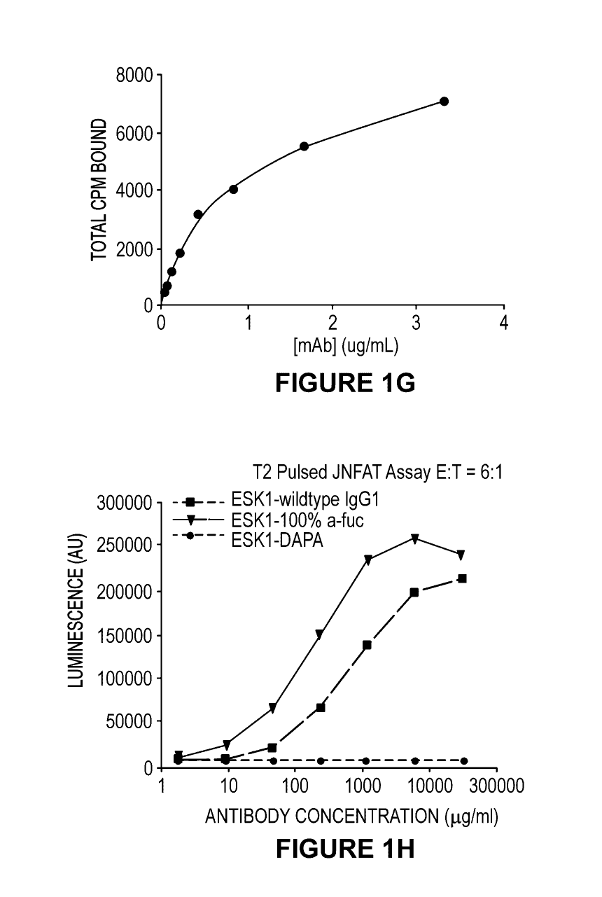

ESKM mAb was produced in MAGE 1.5 CHO cells, with the homogeneous oligosaccharide structure (FIG. 1A) and no detectable fucose or galactose. ESKM had 80% higher affinity for activating human FcγRIIIa (158V variant), 3.5-fold higher affinity for the FcγRIIIa 158F variant, and 50% reduced affinity for inhibitory FcγRIIb (Table 9)

TABLE 9

| | | | | | Ratio of Affinity |
|---|---|---|---|---|---|
| B | Kd +/− SD (nM) | | | | Constants |
| Receptor | ESK1 | | ESKM | | (ESKM/ESK1) |
| Mouse | | | | | |
| FcyRIIb | 32.0 +/− | 0.454 | 62.3 +/− | 7.27 | 0.51 |
| FcyRIV | 3.34 +/− | 0.193 | 2.21 +/− | 0.153 | 1.51 |
| Human | | | | | |
| FcyRI | 0.581 +/− | 0.113 | 0.680 +/− | 0.125 | N.C. |
| FcyRIIa | 105 +/− | 15.5 | 58.3 +/− | 8.80 | 1.81 |
| FcyRIIb | 1338 +/− | 253 | 2644 +/− | 438 | 0.51 |
| FcyRIIIa | 92.6 +/− | 15.0 | 50.4 +/− | 8.35 | 1.84 |
| (158V) | | | | | |
| FcyRIIIa | 19.0 +/− | 2.38 | 5.53 +/− | 0.741 | 3.45 |
| (158F) | | | | | |
| FcRn | 824 +/− | 102 | 780 +/− | 97.5 | N.C. |

Importantly, ESKM affinity for FcRn was unchanged (FIGS. 1B and 1C). Similarly, ESKM had 51% higher affinity for activating mouse FcγRIV, and half the affinity for inactivating mouse FcγRIIb (FIGS. 1B, 1D and 1E). Changes in Fc glycosylation pattern should not be expected to affect antigen binding, and indeed, avidity of ESKM against WT1+ HLA-A0201+ JMN cells was nearly identical to the native ESK1 (0.2-0.4 nM) (FIGS. 1F and 1G).

ESKM showed enhanced reverse signaling through FcγRIIIA (CD16A) compared to wildtype ESK1, indicating improved binding interaction. An approximate 5-fold decrease in EC50 was observed with ESKM relative to wildtype ESK1 (EC50 values: 0.17 for ESKM; 0.88 for ESK1 wildtype; >10000 for ESK1 DAPA) (FIG. 1H).

### ADCC Mediated by ESKM In Vitro.

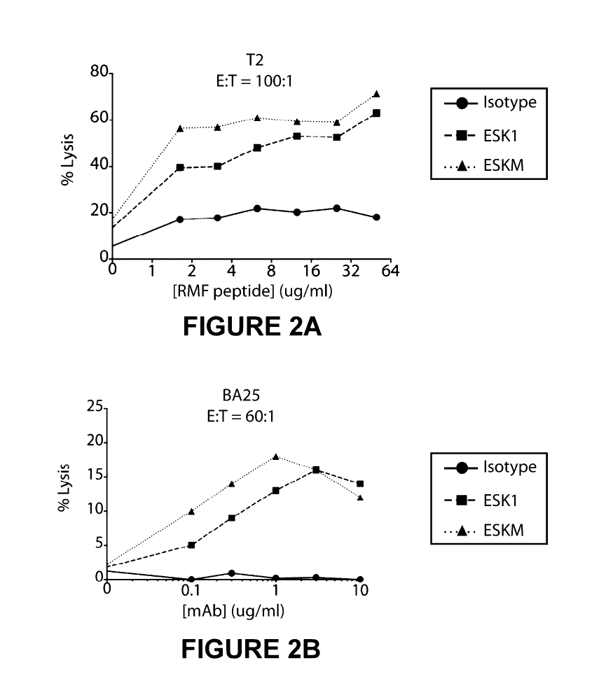

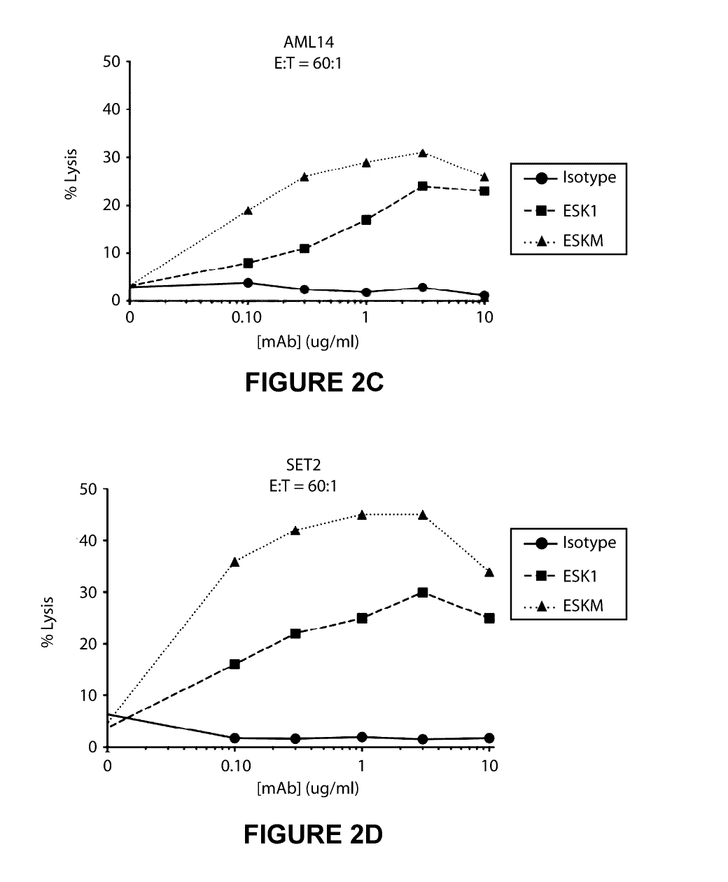

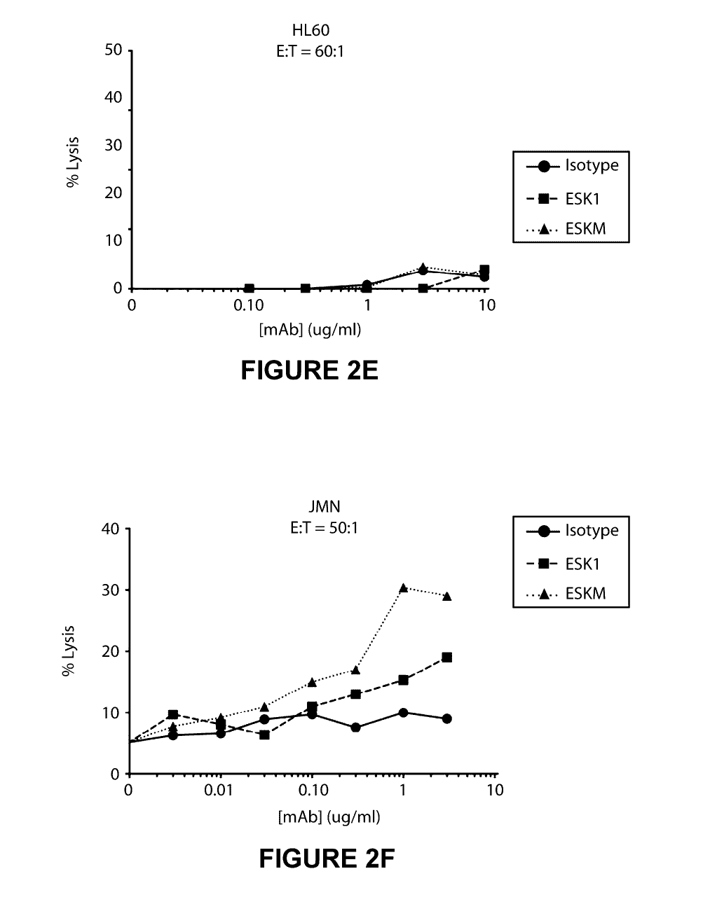

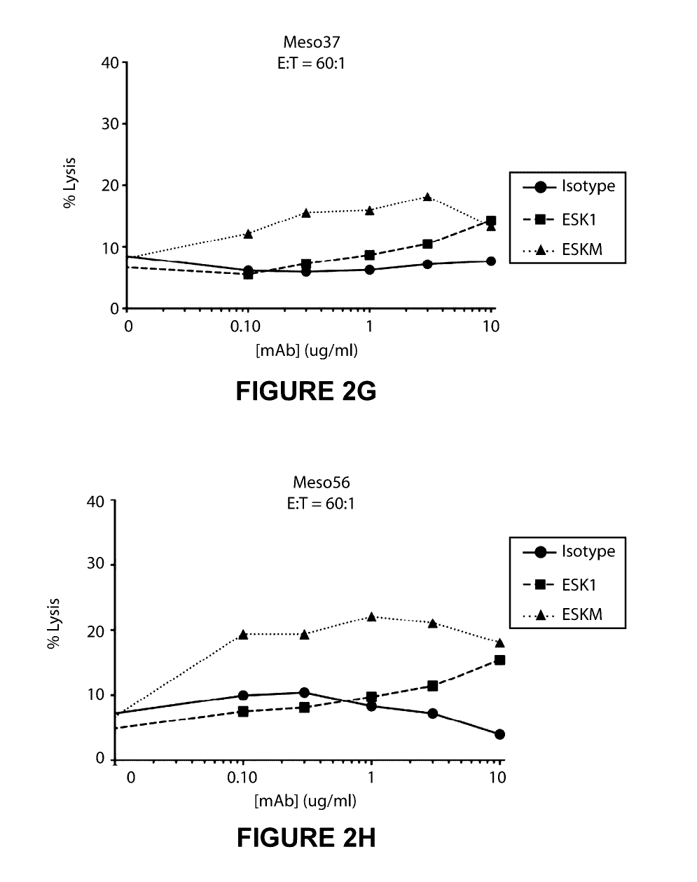

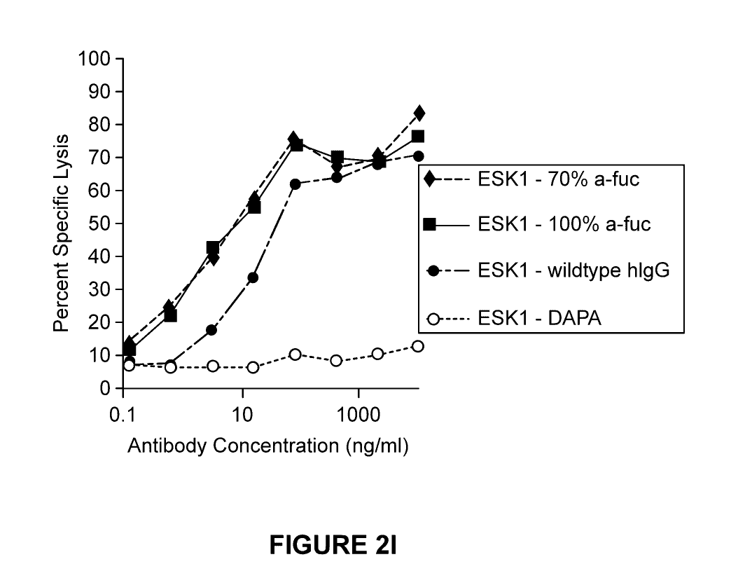

The relationship of cell surface antigen density with ESKM ADCC efficacy was investigated using T2, a TAP-deficient cell line that expresses HLA-A0201, but does not present peptides through the ER pathway, and thus can be loaded with exogenous peptides for presentation in a dose-dependent manner. To determine whether ESKM could better mediate ADCC against cells with low antigen density, the dose of ESK1 and ESKM mAbs was fixed and tested against T2 cells loaded with titrated RMF peptide. Both antibodies were effective against T2 cells pulsed with high peptide concentrations (achieving 40-50% specific lysis), but ESKM was able to mediate greater ADCC against cells with fewer RMF/A2 complexes (FIG. 2A).

The in vitro ADCC activity of ESK1 and ESKM against cell lines presenting a range of levels of cell surface RMF/A2 was determined (Dao T et al. Science translational medicine 2013; 5(176):176ra33). ESKM showed both increased potency and efficacy against six leukemia and mesothelioma cell lines in an HLA-A2-restricted manner. ESKM effectively mediated ADCC against BA-25, an acute lymphoblastic leukemia (ALL) cell line expressing approximately 1000-2000 RMF/A2 targets per cell; both antibodies were similarly effective at ADCC at concentrations above 1 μg/mL, but ESKM was more potent at concentrations down to 100 ng/ml of mAb (FIG. 2B). Against AML-14 and SET2 acute myeloid leukemia (AML) cell lines, which both bind ˜5000 mAb per cell, ESKM mediated higher specific cell lysis than ESK1 at the highest antibody concentrations, and showed cytolytic efficacy down to doses as low as 100 ng/ml (FIG. 1C-1D). Further, the maximal specific lysis achieved against the AML cell lines was generally twice that shown against BA25 (30-45% vs 18%), supporting the hypothesis that increased RMF/A2 levels lead to improved mAb efficacy, regardless of the Fc construct. As was shown previously for ESK1 (Dao T et al. Science translational medicine 2013; 5(176):176ra33), ESKM did not kill leukemia cells not expressing HLA-A2 (FIG. 1E). Further, ESKM mediated higher specific lysis at nearly all doses tested against 3 HLA-A0201+ mesothelioma cell lines: JMN (FIG. 1F), Meso-37 (FIG. 1G) and Meso-56 (FIG. 1H). These data show that ESKM is both more potent—as illustrated by its ability to kill cells with lower mAb concentrations and fewer cell surface targets—and more effective than ESK1, as demonstrated by higher specific lysis attained at equal concentrations.

The effect of different levels of a-fucosylation was evaluated. Both 70% fucose-reduced and 100% fucose-reduced ESKM resulted in greater ADCC activity than the wildtype ESK1 IgG1. An approximate 6-fold decrease in EC50 was observed with ESKM relative to wildtype ESK1 (EC50 values: 2.9 for 100% a-fucosylated ESKM; 3.9 for 70% a-fucosylated ESKM; 18.9 for ESK1 wildtype; >10000 for ESK1 DAPA) (FIG. 21).

### Potency of ESKM Against Human Mesothelioma and Leukemia Models in Mice.

It was previously reported that ESKM is effective at a low dose against bcr/abl+ BV173 ALL in a NSG mouse model. Data from several in vitro and in vivo experiments provided strong evidence that ADCC was the dominant mechanism of therapeutic action of the ESK1 mAb, even in mice lacking functional NK-cells, which might be expected to provide substantial effector function (Dao T et al. Science translational medicine 2013; 5(176):176ra33). To investigate whether ESKM offered a consistent and significant improvement over native ESK1 in vivo in mice with more complete effector cell repertoire, SCID mice, which have intact NK cell functionality, were used, and both antibodies in treatment of human JMN mesothelioma were investigated. Human Fc can engage murine FcγRIV (Pietzsch J et al. Proceedings of the National Academy of Sciences of the United States of America 2012; 109(39): 15859-64), therefore murine NK cells should serve as potent effectors in vivo; as ESKM has enhanced binding to murine FcγRIV, it was expected to be more efficacious than the native mAb in this model. Mice were engrafted with luciferase+ JMN mesothelioma cells in the intraperitoneal cavity (simulating this serosal cavity cancer). To determine the relative abundance of effector cell populations in the intraperitoneal cavity, extracted cells with common murine immunophenotyping markers (Lai L et al. J Immunol 1998; 160(8):3861-8) were analyzed. SCID mice contain intraperitoneal macrophages, neutrophils and NK-cells. Intraperitoneal cells were isolated from mice (n=3 each strain) and analyzed by multi-color flow cytometry. Cell type was determined by the indicated markers, and quantified as percentage of total leukocytes isolated. (Table 10)

TABLE 10
Cells (% of parent population +/− SD)

| Parent | | Marker | | | |
|---|---|---|---|---|---|
| Population | Cell type | Phenotype | BALB/c | C817 SCID | NOG |
| CD11b+ | Granulocyte | GR-1⁺ F4/80⁻ | 46.6 +/− 28.3 | 7.45 +/− 2.71 | 0.215 +/− .137 |
| | Macrophage | GR-^{low}F4/80⁺ | 10.5 +/− 6.87 | 19.0 +/− 1.29 | 96.3 +/− 1.06 |
| | Monocyte | GR-1⁻ F4/80⁻ | 39.2 +/− 21.1 | 70.2 +/− 2.73 | 1.25 +/− 0.562 |
| CD11b− | NK cell | NKp46⁺ | 1.68 +/− 0.250 | 36.2 +/− 12.0 | 0.278 +/− 0413 |
| | | CD3B⁻ | | | |

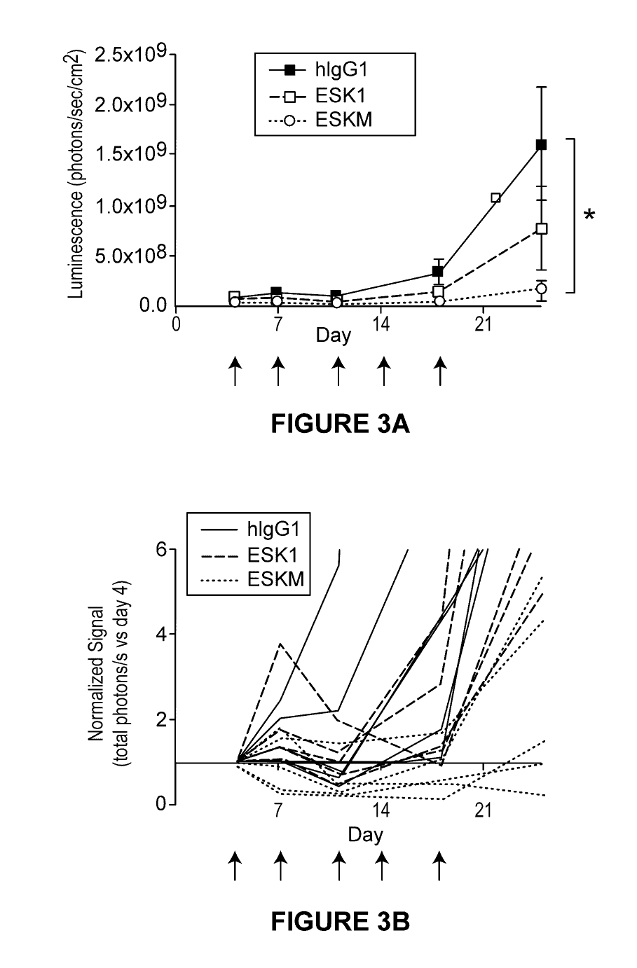

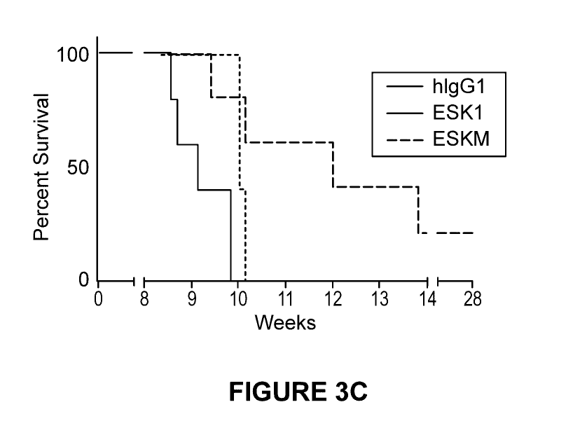

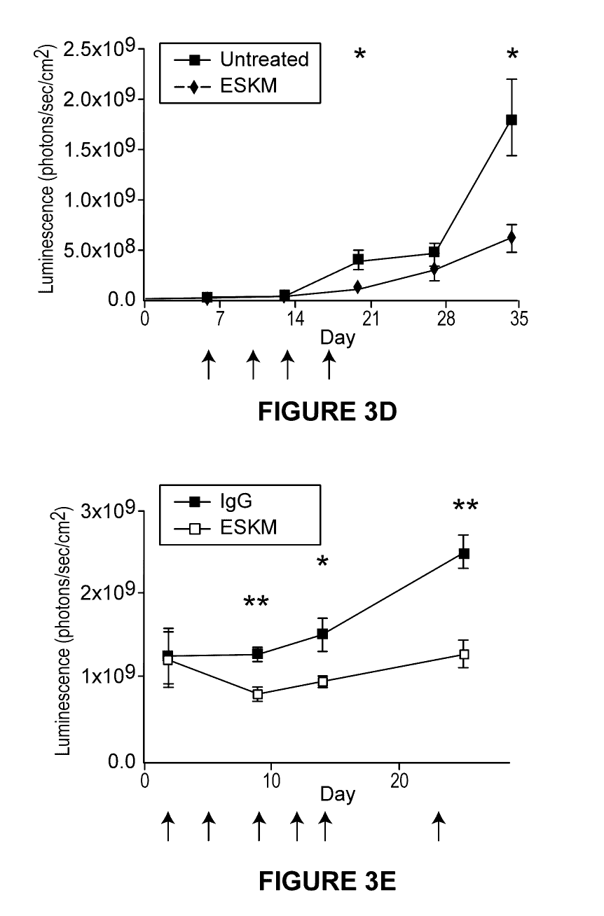

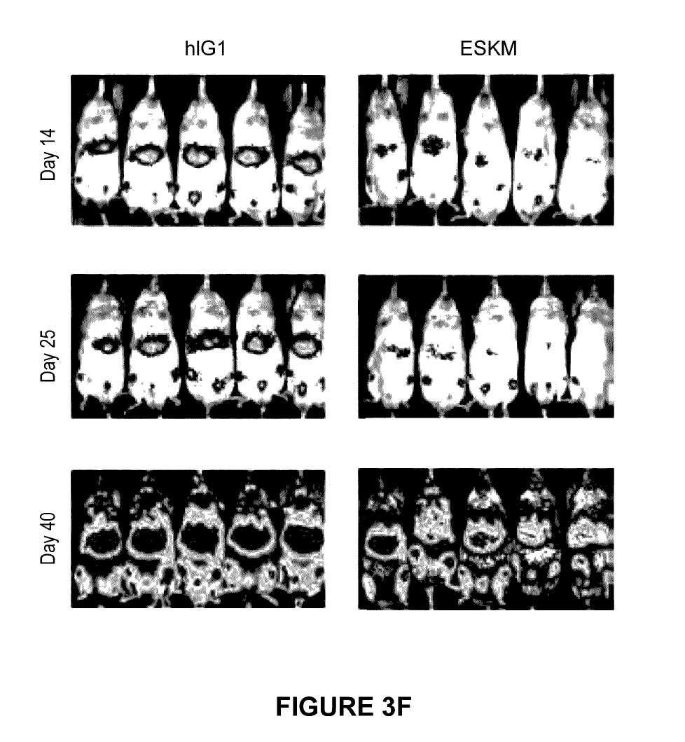

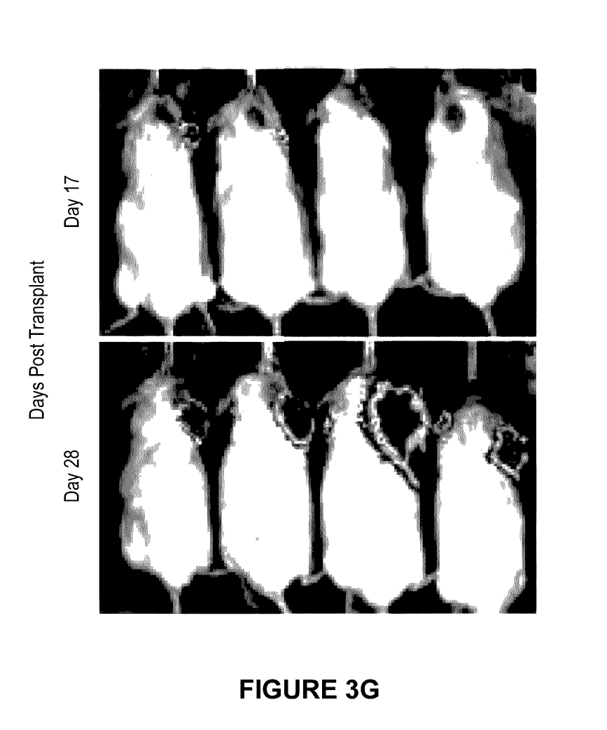

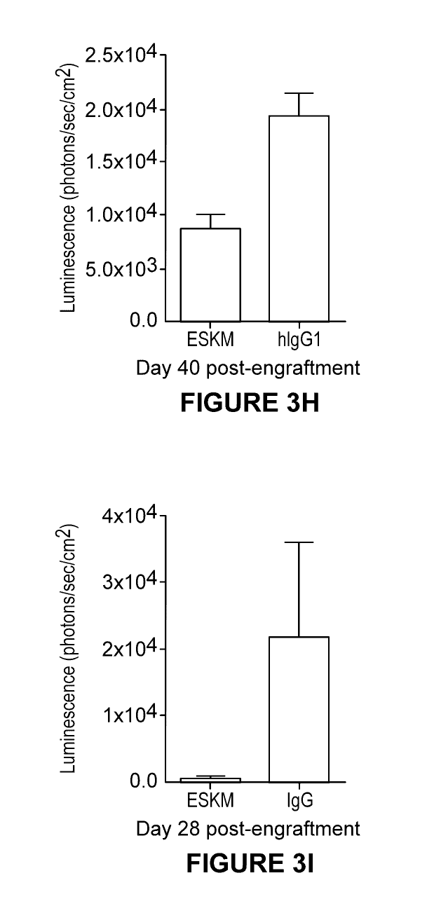

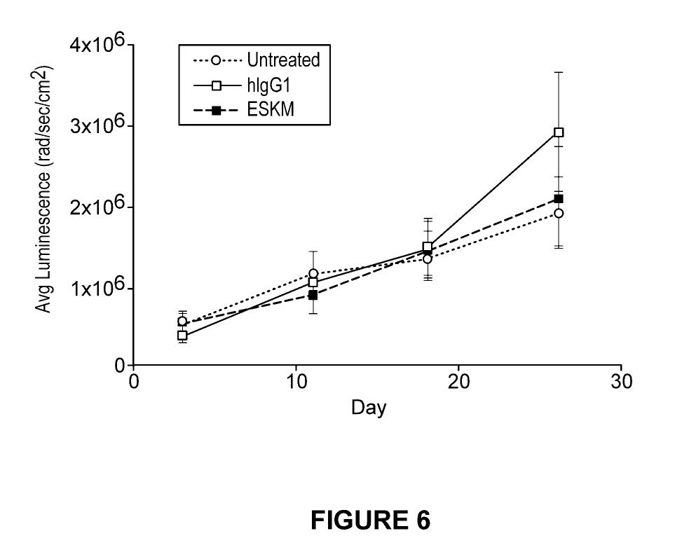

This flow cytometry analysis also confirmed the presence of murine monocytes, macrophages and NK cells, but lack of B- and T-cells in the spleen and peripheral blood, as expected (Bosma M J and Carroll A M. Annual review of immunology 1991; 9:323-50). Biweekly 50 μg treatment with ESKM was more effective than ESK1 against intraperitoneal JMN (FIG. 3A). Further, ESKM was able to reduce tumor burden during the treatment course, whereas ESK1 merely slowed growth (FIG. 3B). ESKM treated mice survived significantly longer than isotype-treated, and had improved survival over ESK1-treated groups (FIG. 3C). In a third experiment at the same dose and schedule, neither antibody construct showed efficacy against intraperitoneal JMN in NOG mice (FIG. 6), which lack NK-cells and intraperitoneal neutrophils, indicating that these cell populations likely play an important role in efficacy in these models. These studies provide further evidence that ESKM is a more potent mAb construct.

ESKM was also investigated in the luciferase+ SET2 mouse model of AML. The SET2 cell line grew much faster than BV173 in the NSG mouse model and disseminated throughout the mouse bone marrow. ESKM was able to significantly reduce tumor growth (FIG. 3D). To further address the clinical utility of ESKM, a fresh human pre-B-cell ALL derived from a CNS-relapse was engrafted into NSG mice. ESKM significantly reduced initial leukemia burden and slowed leukemia outgrowth (FIGS. 3E and 3F). Leukemia relapsed after treatment was stopped (FIG. 3F), allowing for leukemia cells to be collected from the bone marrow and transplanted to new animals to assess outgrowth from remaining progenitors (FIG. 3G). Total bone marrow signal in ESKM-treated mice was lower at time of transplant (FIG. 3H), but equal numbers of ESKM-treated and isotype-treated bone marrow cells were engrafted into recipient animals. Subcutaneous leukemia tumors from isotype-treated leukemia cells grew 20 times faster than from ESKM-treated cells (FIG. 3I).

### Pharmacokinetics and Biodistribution of ESK1 and ESKM

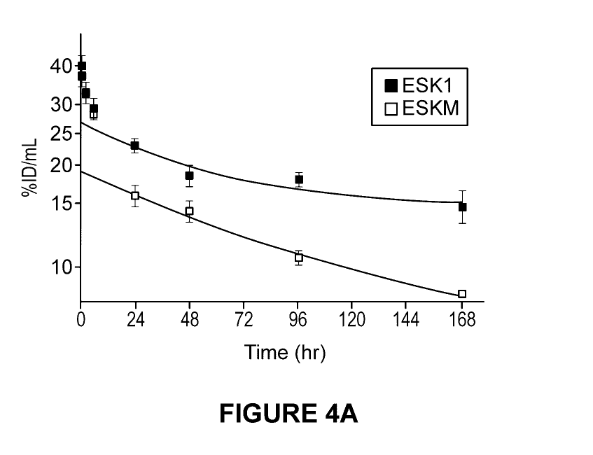

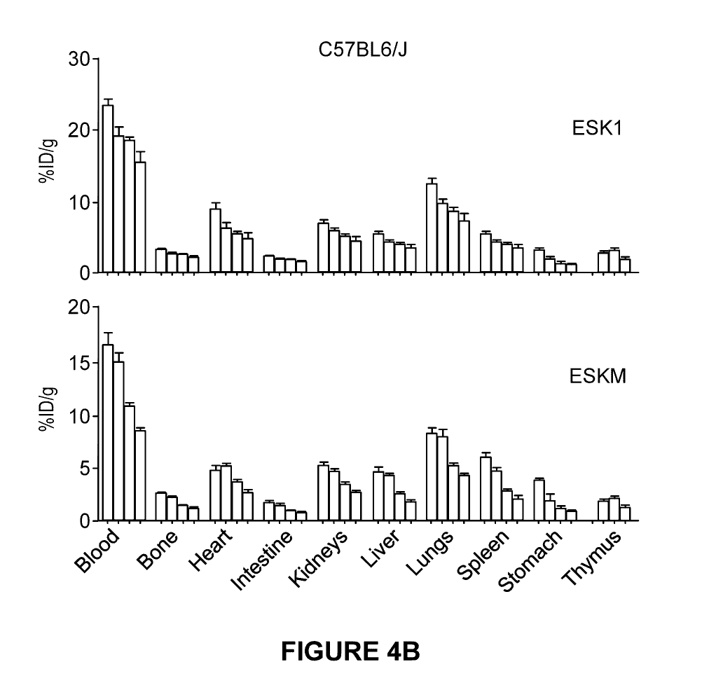

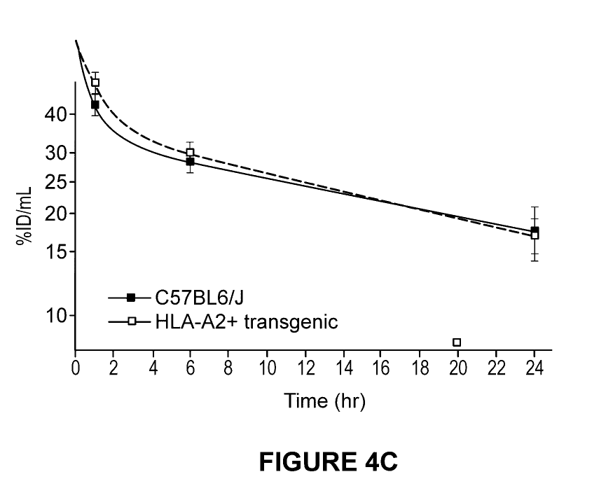

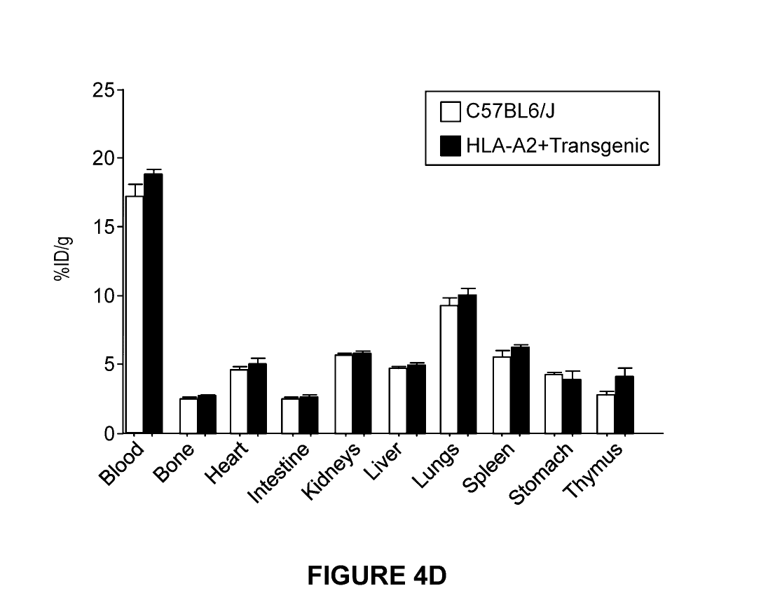

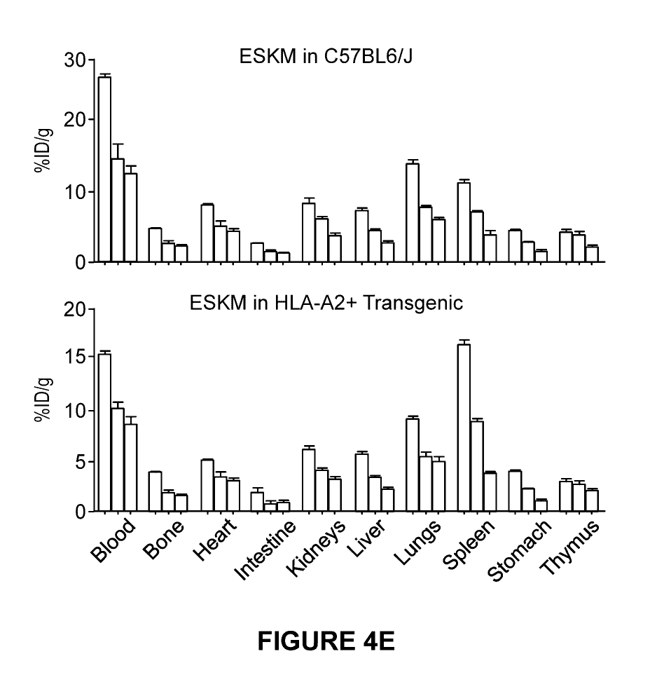

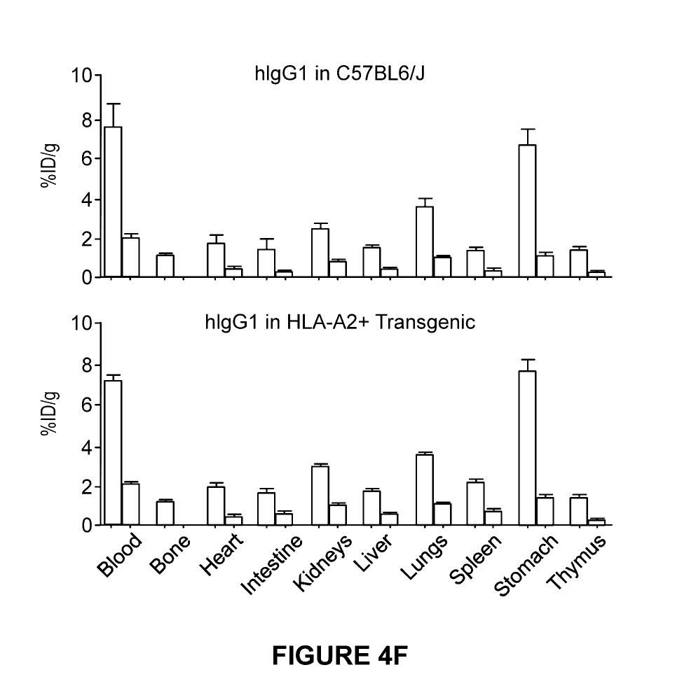

Altering Fc glycosylation could potentially change pharmacokinetic properties of the mAb, thereby affecting its therapeutic utility. To determine mAb pharmacokinetics, trace ¹²⁵I-labeled antibodies were injected intravenously into C57 BL6/J mice and blood levels of mAb were measured over 7 days. Both ESK1 and ESKM exhibited biphasic clearance common of monoclonal antibodies, with initial tissue distribution and an alpha half-life of 1.1-2.4 hr, followed by a slower beta half-life of several days (FIG. 4A). ESKM had a shorter beta half-life than ESK1 (4.9 days vs 6.5 days). The biodistribution patterns of the antibodies were determined using the same radiolabeled constructs. Both antibodies displayed similar patterns of organ distribution and clearance (FIG. 4B).

While increased FcγRIV binding or manose receptor binding could create a sink for ESKM in FcγRIV-expressing tissues, no increase in ESKM distribution to the liver, spleen, thymus or bone marrow was found that could account for the shortened serum half-life.

As the ESKM antibody targets a human-specific epitope, the C57BL6/J mouse model cannot recapitulate possible on-target binding to normal tissues that could alter antibody pharmacokinetics and biodistribution. Therefore, a transgenic mouse model based on the C57BL6/J background that expresses human HLA-A201 driven by a lymphoid promoter was used. The 9-mer RMF sequence is identical in human and mouse, and therefore this transgenic model could recapitulate antigen presentation of the RMF/HLA-A0201 epitope in various healthy organs. There was no difference between wild-type and HLA-A0201 transgenic mice in blood pharmacokinetics of ESKM, indicating that there was no significant antibody sink (FIG. 4C). Further, at a therapeutic dose of antibody (100 μg), there was no difference in biodistribution in transgenic compared to wild-type mice (FIG. 4D).

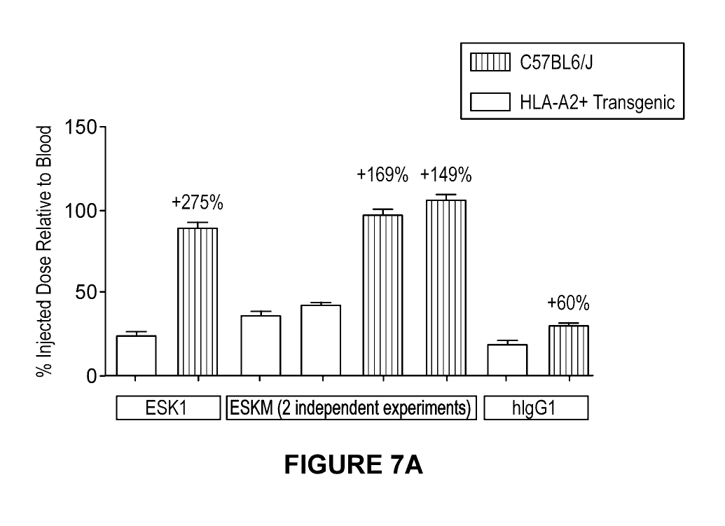

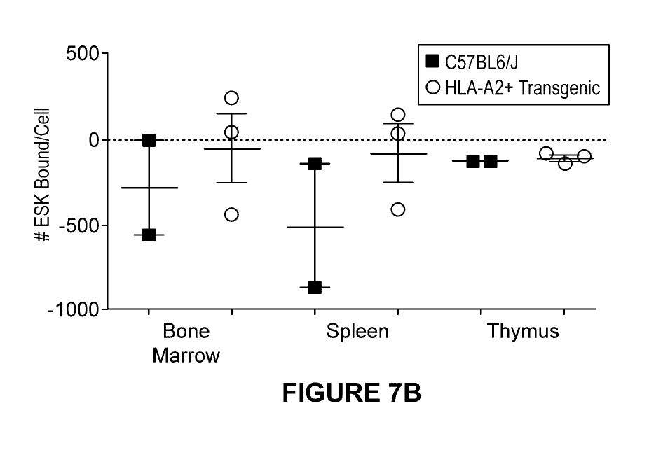

At low doses of trace-labeled ESKM (2 μg) there was a small yet detectable increase in uptake of antibody in the spleen of transgenic mice compared to wild-type (FIG. 4E). The additional binding in HLA-A0201 transgenic spleens accounted for only 16 ng of antibody. This small uptake could be due to RMF presentation in HLA-A0201+ cells, or to an unknown cross-reacting epitope. Splenic uptake was not due to Fc glycosylation pattern alone, as the native ESK1 mAb also showed increased uptake in transgenic spleens at 24 hours (FIG. 7A). Additionally, increased spleen uptake in transgenic mice appeared to be partly related to strain differences in clearance, as the isotype control antibody also showed 57% increased splenic uptake at 24 hours in transgenic compared to wild-type mice (FIG. 4F). Further, no binding of ESK1 to cells isolated from A0201 transgenic mouse spleen cells was observed by either flow cytometry (Dao T et al. Science translational medicine 2013; 5(176):176ra33) or specific binding assay with ¹²⁵I-labeled ESK1 (FIG. 7B), suggesting that if a cross-reacting epitope was present, it was not expressed in detectable amounts on a specific cell type.

### Toxicity of ESKM in HLA-A0201 Transgenic Mouse Model

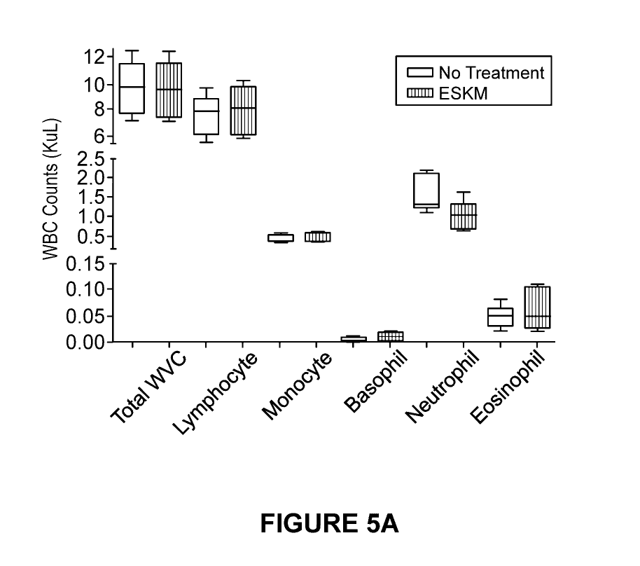

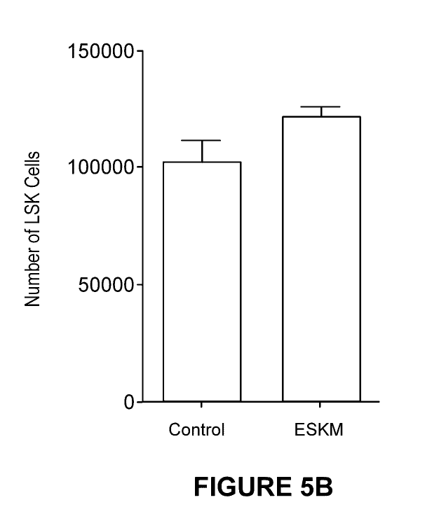

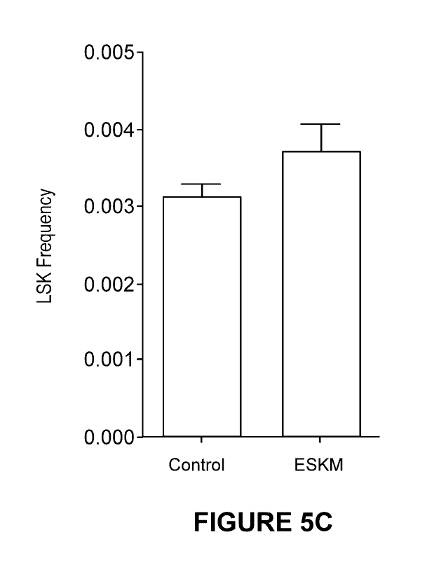

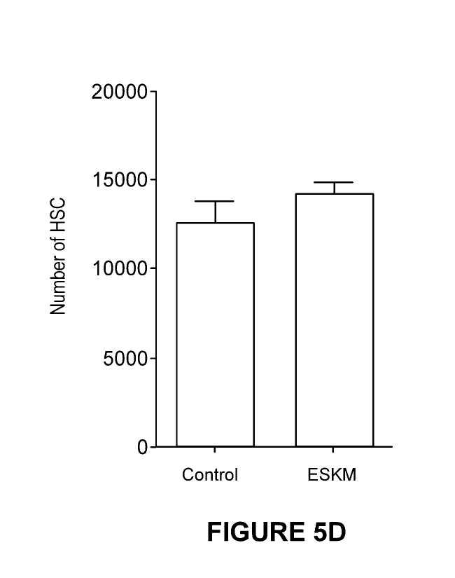

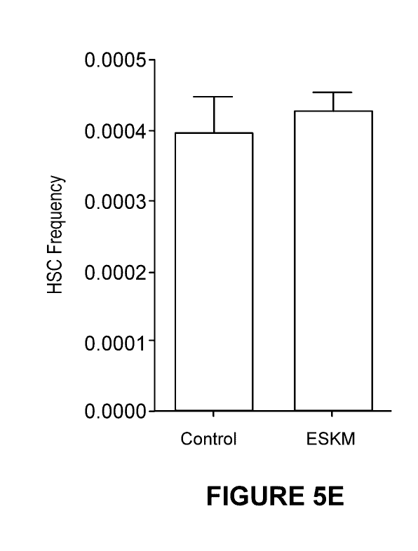

WT1 is reported to be expressed in hematopoietic stem cells (HSC) (Ariyaratana S and Loeb D M. Expert reviews in molecular medicine 2007; 9(14):1-17), so the C57 BL6/J transgenic mouse model with human HLA-A0201 driven by a lymphoid promoter provides an opportunity to assess possible toxicity against progenitor cells in the hematopoietic compartment that, given the high potency of ESKM, might occur even at low epitope density. To assess toxicity, white blood cell and bone-marrow cell counts were measured one day after the final of two therapeutic doses of ESKM or isotype control mAb on the same schedule as previously described therapy experiments (Dao T et al. Science translational medicine 2013; 5(176):176ra33). There were no differences in total white blood cell count, or lymphocytes, neutrophils, monocytes, eosinophil or basophil cell counts (FIG. 5A). Within the bone-marrow compartment, equivalent absolute number and frequency of hematopoietic stem cell (HSC) progenitors (LSK: Lineage^{lo}, c-kit⁺, Sca1⁺) (FIG. 5B-5C) and HSCs (CD150hi, CD48⁻, LSK) (FIG. 5D-5E) were found in the ESKM treated and isotype control-treated mice.

Finally, gross and microscopic pathology of lymphoid and major organs of A0201 transgenic mice treated with ESKM or isotype mAb on the same schedule were assessed. No striking differences between ESKM- and isotype-treated groups were observed by a trained hematopathologist (Table 11). Almost all of the target organs were present with good histology.

TABLE 11

| | | | | | | | | bone |
|---|---|---|---|---|---|---|---|---|
| | spleen | thymus | liver | kidney | lung | GI | heart | marrow |
| hIgG1-1 | Nml | Nml | Nml | Nml | Nml | Nml | Nml | Nml |
| hIgG1-2 | Nml | N/A* | Nml | Nml | Nml | Nml | Nml | Nml |
| hIgG1-3 | Nml | Nml | Nml | Nml | Nml | Nml | Nml | Nml |
| hIgG1-4 | Nml | Nml | Nml | Nml | Nml | Nml | Nml | Nml |
| hIgG1-5 | Nml | Nml | Nml | Nml | Nml | Nml | Nml | Nml |
| ESKM-1 | Nml | Nml | Nml | Nml | Nml | Nml | Nml | Nml |
| ESKM-2 | Nml | Nml | Nml | Nml | Nml | Nml | Nml | Nml |
| ESKM-3 | Nml | Nml** | Nml | Nml | Nml | Nml | Nml | Nml |
| ESKM-4 | Nml | Nml | Nml | Nml | Nml | Nml | Nml | Nml |
| ESKM-5 | Nml | Nml | Nml | Nml | Nml | Nml | Nml | Nml |

Nml—Normal
*Thymus specimen is missing from specimen IgG-2A

The bone marrow sections of both groups showed trilineage hematopoiesis with adequate maturation of the myeloid and erythroid lineages. The megakaryocytes were adequate in number with normal morphology. Thymus sections showed a well-defined cortex and medulla with few Hassall's corpuscles, which is normal for rodent histology. The kidney sections showed no pathologic findings such as glomerulosclerosis, congestion, or inflammation. Liver sections showed normal lobular architecture without congestion or inflammation. All spleen sections showed a normal distribution of red and white pulp. Occasional scattered megakaryocytes were seen in the red pulp consistent with extramedullary hematopoiesis (Cesta M F. Toxicologic pathology 2006; 34(5):455-65). Heart sections of the control groups and treatment groups had normal myocytes without inflammation or fibrosis. Small intestine sections showed normal villi and crypts, and incidentally sampled pancreas sections for some of the control and treatment groups showed no pathologic findings. Finally, the lung sections of both treatment groups were unremarkable.

The generation of TCRm antibodies allows use of the mAb to target cell-surface fragments of intracellular proteins, provided that they are processed and presented on MHC class I molecules. ESK1 was the first TCRm antibody reported against a peptide derived from WT1, an important oncogene expressed in a wide variety of cancers, but not normal adult tissues. WT1 appears to be expressed in leukemic stem cells (Ariyaratana S and Loeb D M. Expert reviews in molecular medicine 2007; 9(14):1-17), raising the possibility that the mAb could ultimately eliminate clonogenic leukemia cells in patients. Other therapeutic TCRm mouse antibodies, human ScFv and Fab fragments have been previously described (Epel M et al. European journal of immunology 2008; 38(6):1706-20; Wittman V P et al. J Immunol 2006; 177(6):4187-95; Klechevsky et al. Cancer research 2008; 68(15):6360-7; Verma B, et al. J Immunol 2010; 184(4):2156-65; Sergeeva et al. Blood 2011; 117(16):4262-72). However, ESK1 is the first and only fully human therapeutic TCRm mAb reported.

The features of the RMF/A2 epitope, especially the low levels of expression on the cell surface, require selection of a highly potent and effective ESK1 construct. The ESK1 construct was improved by altering Fc glycosylation as a means to enhance ADCC, the major mechanism of ESK1 action in vitro and in vivo (Dao T et al. Science translational medicine 2013; 5(176):176ra33). Several ADCC-enhanced mAbs to highly expressed cell-surface antigens, produced, either by glyco-engineering or point mutations, are in clinical trials in the U.S. with promising results (Kubota T et al. Cancer science 2009; 100(9):1566-72; Ishida T et al. Journal of clinical oncology: official journal of the American Society of Clinical Oncology 2012; 30(8):837-4; Subramaniam J M et al. Drugs 2012; 72(9):1293-8). The ESKM mAb had a homogeneous glycosylation pattern lacking N-linked fucose and with terminal hexose (mannose and/or glucose) structure. This engineering strategy modulates mAb binding to Fcγ receptors in two ways: a higher affinity for activating human FcγRIIIa (and murine FcγRIV) increases ADCC activity; while diminished affinity for both human and murine FcγRIIb should reduce inhibitory receptor activation. As expected, ESKM was both more potent and effective in vitro even at very low epitope density.

ESKM was also more effective than ESK1 in vivo, and was able to treat peritoneal mesothelioma in SCID mice, modeling the clinical situation. Three of 5 animals displayed absolute reduction in tumor burden over the two-week treatment course, whereas none of the ESK1-treated mice achieved more than a slowing of initial tumor growth. After termination of therapy, ESKM-treated mice survived longer than ESK1 treatment groups, with 1 of 5 animals surviving without disease. Further, ESKM significantly slowed leukemia growth of disseminated SET2, an AML cell line with much more aggressive in vivo leukemia growth kinetics than BV173, and a fresh patient-derived pre-B-cell ALL in xenograft models. In the fresh ALL model, tumor relapsed after mAb therapy was stopped, but leukemia cells extracted from the bone marrow of ESKM-treated mice and transplanted as subcutaneous tumors showed minimal outgrowth. This suggests that ESKM may target a progenitor population of leukemia cells, which is consistent with the hypothesis that WT1 expression in HSCs could allow ablation of this population. However, cells collected from the bone marrow were not phenotyped and sorted, so the exact cell population targeted was not determined. These data provide further evidence that ESKM is a potent agent in diverse mouse models of human cancer.

ESKM therapy was not effective against peritoneal mesothelioma in NSG or NOG mice, which lack NK-cells, though naked ESK1 did previously show potent activity against a disseminated leukemia model in these mice. This discrepancy could be due both to the tumor model—leukemia cells could have different sensitivity to effector-mediated cytotoxicity—and to the availability of effector cells in the NSG/NOG model. Access to ESK-bound target cells is likely more optimal in the circulation and hematopoietic compartments, where the leukemia grew, than in the peritoneal cavity; further, assays indicated that the intraperitoneal cavity of NOG mice contained predominately macrophages, while neutrophils were present in the blood and spleen. The marked improvement in efficacy with ESKM in SCID mice indicated that NK cells and/or monocytes (both with FcγRIV) are important to therapy in this model.

Altering Fc glycosylation could potentially change pharmacokinetic properties of the mAb through a number of mechanisms, including: altered FcRn binding and antibody recycling, modified binding to circulating effector cells, and differential engagement with clearance mechanisms, such as mannose receptors. Similar afucosylated, Fc-modified antibodies with improved ADCC have been investigated in pharmacokinetic studies in vivo (Gasdaska J R et al. Molecular immunology 2012; 50(3):134-41, Junttila T T et al. Cancer research 2010; 70(11): 4481-9). ESKM had nearly identical biodistribution to ESK1, but a shortened blood half-life. No change in biodistribution pattern that could account for this altered half-life was seen. IgG half-life is regulated by the neonatal Fc receptor, FcRn (Raghavan M and Bjorkman P J. Annual review of cell and developmental biology 1996; 12:181-220; Roopenian D C and Akilesh S. Nature reviews Immunology 2007; 7(9):715-25); however, ESKM had identical affinity for FcRn as ESK1. The altered pharmacokinetics is possibly due to interaction with mannose receptor on macrophages, a known mechanism of glycoprotein clearance (Allavena P et al. Critical reviews in immunology 2004; 24(3):179-92; Lee S J et al. Science 2002; 295(5561):1898-901; Stahl P D. Curr Opin Immunol 1992; 4(1):49-52).

Since ESK mAbs target a human HLA-specific epitope, the human HLA-A0201+ transgenic mouse strain was utilized for toxicology studies. WT1 is reportedly expressed in HSCs, yet a therapeutic dose of ESKM that cleared leukemia in the models had no effect on LSK cells or early HSCs. Further, after this same treatment schedule, organ histology was normal. Importantly, ESKM did not affect the architecture or cell coverage in the bone marrow, thymus or spleen, where WT1+ HSCs could be expected, and where HLA-A0201 expression is highest because the transgene is driven by a lymphoid promoter. There was also no observed pathology in the kidney, where WT1 expression might be expected in mature podocytes.

ESKM has moderately decreased half-life yet increased potency and broader applicability. The potential enhanced efficacy against tumors expressing fewer RMF/A2 sites could expand the number of patients and cancer types eligible for this therapy as well as increase efficacy. In addition, the MAGE 1.5 CHO engineering technology generates mAbs that effectively engage FcγRIIIa (CD16), regardless of amino acid 158 polymorphism. Carriers of CD16-158F are less responsive than CD16-158V/V individuals to human IgG1 therapeutics such as rituximab and trastuzumab (Cartron G et al. Blood 2002; 99(3):754-8, Musolino A et al. Journal of clinical oncology: official journal of the American Society of Clinical Oncology 2008; 26(11):1789-96). WT1 is expressed in multiple cancers (Sugiyama H. Japanese journal of clinical oncology 2010; 40(5):377-87), making RMF/A2 a potential therapeutic target for many indications. In preclinical models, efficacy against bcr/abl+ ALL and B-ALL (Dao T et al. Science translational medicine 2013; 5(176):176ra33), and here, AML and mesothelioma xenografts, was shown. In summary, ESKM is a potent therapeutic mAb against a widely expressed oncogenic target with a restricted normal cell expression profile, and has shown efficacy against multiple human tumor models in mice.

Although the foregoing invention has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

1. An antibody comprising:
(A) a heavy chain (HC) variable region comprising HC-CDR1, HC-CDR2 and HC-CDR3 respectively, comprising amino acid sequences SEQ ID NOS: 2, 3, and 4; 18, 19 and 20; 34, 35, and 36; 50, 51, and 52; 66, 67, and 68 or 82, 83, and 84; and a light chain (LC) variable region comprising LC-CDR1, LC-CDR2 and LC-CDR3 respectively, comprising amino acid sequences SEQ ID NOS: 8, 9 and 10; 24, 25 and 26; 40, 41 and 42; 56, 57 and 58; 72, 73 and 74 or 88, 89 and 90; or
(B) a V_{H} and V_{L} comprising the amino acid sequence of SEQ ID NO: 14 and SEQ ID NO: 16; 30 and 32; 46 and 48; 62 and 64; 78 and 80 or 94 and 96, respectively,
wherein said antibody has reduced fucose or galactose.

2. The antibody of claim 1, comprising a heavy chain (HC) variable region comprising HC-CDR1, HC-CDR2 and HC-CDR3 respectively, comprising amino acid sequences SEQ ID NOS: 2, 3, and 4; and a light chain (LC) variable region comprising LC-CDR1, LC-CDR2 and LC-CDR3 respectively, comprising amino acid sequences SEQ ID NOS: 8, 9 and 10.

3. The antibody of claim 1, comprising a heavy chain (HC) variable region comprising HC-CDR1, HC-CDR2 and HC-CDR3 respectively, comprising amino acid sequences SEQ ID NOS: 18, 19 and 20; and a light chain (LC) variable region comprising LC-CDR1, LC-CDR2 and LC-CDR3 respectively, comprising amino acid sequences SEQ ID NOS: 24, 25 and 26.

4. The antibody of claim 1, comprising a heavy chain (HC) variable region comprising HC-CDR1, HC-CDR2 and HC-CDR3 respectively, comprising amino acid sequences SEQ ID NOS: 34, 35, and 36; and a light chain (LC) variable region comprising LC-CDR1, LC-CDR2 and LC-CDR3 respectively, comprising amino acid sequences SEQ ID NOS: 40, 41 and 42.

5. The antibody of claim 1, comprising a heavy chain (HC) variable region comprising HC-CDR1, HC-CDR2 and HC-CDR3 respectively, comprising amino acid sequences SEQ ID NOS: 50, 51, and 52; and a light chain (LC) variable region comprising LC-CDR1, LC-CDR2 and LC-CDR3 respectively, comprising amino acid sequences SEQ ID NOS: 56, 57 and 58.

6. The antibody of claim 1, comprising a heavy chain (HC) variable region comprising HC-CDR1, HC-CDR2 and HC-CDR3 respectively, comprising amino acid sequences SEQ ID NOS: 66, 67, and 68; and a light chain (LC) variable region comprising LC-CDR1, LC-CDR2 and LC-CDR3 respectively, comprising amino acid sequences SEQ ID NOS: 72, 73 and 74.

7. The antibody of claim 1, comprising a heavy chain (HC) variable region comprising HC-CDR1, HC-CDR2 and HC-CDR3 respectively, comprising amino acid sequences SEQ ID NOS: 82, 83, and 84; and a light chain (LC) variable region comprising LC-CDR1, LC-CDR2 and LC-CDR3 respectively, comprising amino acid sequences SEQ ID NOS: 88, 89 and 90.

8. The antibody of claim 1, comprising a light chain consisting essentially of the amino acid sequence of SEQ ID NO: 100 and a heavy chain consisting essentially of the amino acid sequence of SEQ ID NO: 101.

9. The antibody of claim 1, wherein the fucose content or galactose content of said antibody is reduced by at least 70% compared to wildtype antibody.

10. The antibody of claim 9, wherein the fucose content or galactose content of said antibody is reduced by 100% compared to wildtype antibody.

11. The antibody of claim 1, wherein said antibody specifically binds to WT-1 peptide RMFPNAPYL (SEQ ID NO: 1) in conjunction with HLA/A2.

12. The antibody of claim 1, wherein said antibody exhibits between 50-100% (80%) higher affinity for activating human FcγRIIIa (158V variant) than normally glycosylated antibody, has 3-4-fold (3.5-fold) higher affinity for a FcγRIIIa 158F variant than normally glycosylated antibody, and has between 30 and 70% (50%) reduced affinity for inhibitory FcγRIIb than normally glycosylated antibody.

13. The antibody of claim 12, wherein said HLA-A2 is HLA-A0201.

14. An isolated nucleic acid that encodes an antibody of claim 1.

15. A vector comprising a nucleic acid of claim 14.

16. A cell comprising a nucleic acid of claim 14.

17. A cell comprising a vector of claim 14.

18. A kit comprising an antibody of claim 1.

19. A pharmaceutical composition comprising an antibody of claim 1 to 13 and a pharmaceutically acceptable carrier.

20. (canceled)

21. A method for treatment of a subject having a WT1-positive disease, comprising administering to the subject a therapeutically effective amount of an antibody or antigen binding fragment thereof of claim 1 or a pharmaceutical composition thereof.

22. (canceled)

23. The method of claim 21, wherein the WT1-positive disease is a chronic leukemia or acute leukemia or WT1+ cancer.

24. The method of claim 21, wherein the WT1-positive disease is selected from the group consisting of chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma.


SEQUENCE LISTING (102 sequences)

SEQ ID NO: 1 - protein, 9 aa, Artificial Sequence
  Synthetic Polypeptide
RMFPNAPYL

SEQ ID NO: 2 - protein, 10 aa, Artificial Sequence
  Synthetic Polypeptide
GGTFSSYAIS

SEQ ID NO: 3 - protein, 17 aa, Artificial Sequence
  Synthetic Polypeptide
GIIPIFGTANYAQKFQG

SEQ ID NO: 4 - protein, 10 aa, Artificial Sequence
  Synthetic Polypeptide
RIPPYYGMDV

SEQ ID NO: 5 - DNA, 30 bp, Artificial Sequence
  Synthetic Polynucleotide
ggaggcaccttcagcagctatgctatcagc

SEQ ID NO: 6 - DNA, 51 bp, Artificial Sequence
  Synthetic Polynucleotide
gggatcatccctatctttggtacagcaaactacgcacagaagttccagggc

SEQ ID NO: 7 - DNA, 30 bp, Artificial Sequence
  Synthetic Polynucleotide
cggattcccccgtactacggtatggacgtc

SEQ ID NO: 8 - protein, 13 aa, Artificial Sequence
  Synthetic Polypeptide
SGSSSNIGSNYVY

SEQ ID NO: 9 - protein, 7 aa, Artificial Sequence
  Synthetic Polypeptide
RSNQRPS

SEQ ID NO: 10 - protein, 11 aa, Artificial Sequence
  Synthetic Polypeptide
AAWDDSLNGVV

SEQ ID NO: 11 - DNA, 39 bp, Artificial Sequence
  Synthetic Polynucleotide
tctggaagcagctccaacatcggaagtaattatgtatac

SEQ ID NO: 12 - DNA, 21 bp, Artificial Sequence
  Synthetic Polynucleotide
aggagtaatcagcggccctca

SEQ ID NO: 13 - DNA, 33 bp, Artificial Sequence
  Synthetic Polynucleotide
gcagcatgggatgacagcctgaatggtgtggta

SEQ ID NO: 14 - protein, 119 aa, Artificial Sequence
  Synthetic Polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRIPPYYGMDVWGQGTTVTVSS

SEQ ID NO: 15 - DNA, 357 bp, Artificial Sequence
  Synthetic Polynucleotide
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagacggattcccccgtactacggtatggacgtctggggccaagggaccacggtcaccgtctcctca

SEQ ID NO: 16 - protein, 111 aa, Artificial Sequence
  Synthetic Polypeptide
QTVVTQPPSASGTPGQRVTISCSGSSSNIGSNYVYWYQQLPGTAPKLLIYRSNQRPSGVPDRFSGSKSGTSASLAISGPRSVDEADYYCAAWDDSLNGVVFGGGTKLTVLG

SEQ ID NO: 17 - DNA, 333 bp, Artificial Sequence
  Synthetic Polynucleotide
cagactgtggtgactcagccaccctcagcgtctgggacccccgggcagagggtcaccatctcttgttctggaagcagctccaacatcggaagtaattatgtatactggtaccaacagctcccaggaacggcccccaaactcctcatctataggagtaatcagcggccctcaggggtccctgaccgattctctggctccaagtctggcacctcagcctccctggccatcagtgggccccggtccgtggatgaggctgattattactgtgcagcatgggatgacagcctgaatggtgtggtattcggcggagggaccaagctgaccgtcctaggt

SEQ ID NO: 18 - protein, 12 aa, Artificial Sequence
  Synthetic Polypeptide
GDSVSSNSAAWN

SEQ ID NO: 19 - protein, 18 aa, Artificial Sequence
  Synthetic Polypeptide
RTYYGSKWYNDYAVSVKS

SEQ ID NO: 20 - protein, 9 aa, Artificial Sequence
  Synthetic Polypeptide
GRLGDAFDI

SEQ ID NO: 21 - DNA, 36 bp, Artificial Sequence
  Synthetic Polynucleotide
ggggacagtgtctctagcaacagtgctgcttggaac

SEQ ID NO: 22 - DNA, 54 bp, Artificial Sequence
  Synthetic Polynucleotide
aggacatactacgggtccaagtggtataatgattatgcagtatctgtgaaaagt

SEQ ID NO: 23 - DNA, 27 bp, Artificial Sequence
  Synthetic Polynucleotide
ggtcgcttaggggatgcttttgatatc

SEQ ID NO: 24 - protein, 11 aa, Artificial Sequence
  Synthetic Polypeptide
RASQSISSYLN

SEQ ID NO: 25 - protein, 7 aa, Artificial Sequence
  Synthetic Polypeptide
AASSLQS

SEQ ID NO: 26 - protein, 9 aa, Artificial Sequence
  Synthetic Polypeptide
QQSYSTPLT

SEQ ID NO: 27 - DNA, 33 bp, Artificial Sequence
  Synthetic Polynucleotide
cgggcaagtcagagcattagcagctatttaaat

SEQ ID NO: 28 - DNA, 21 bp, Artificial Sequence
  Synthetic Polynucleotide
gctgcatccagtttgcaaagt

SEQ ID NO: 29 - DNA, 27 bp, Artificial Sequence
  Synthetic Polynucleotide
caacagagttacagtacccctctcact

SEQ ID NO: 30 - protein, 121 aa, Artificial Sequence
  Synthetic Polypeptide
QVQLQQSGPGLVKPSQTLSLTCAISGDSVSSNSAAWNWIRQSPSRGLEWLGRTYYGSKWYNDYAVSVKSRITINPDTSKNQFSLQLNSVTPEDTAVYYCARGRLGDAFDIWGQGTMVTVSS

SEQ ID NO: 31 - DNA, 363 bp, Artificial Sequence
  Synthetic Polynucleotide
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacgggtccaagtggtataatgattatgcagtatctgtgaaaagtcgaataaccatcaacccagacacatccaagaaccagttctccctgcagctgaactctgtgactcccgaggacacggctgtgtattactgtgcaagaggtcgcttaggggatgcttttgatatctggggccaagggacaatggtcaccgtctcttca

SEQ ID NO: 32 - protein, 108 aa, Artificial Sequence
  Synthetic Polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIKR

SEQ ID NO: 33 - DNA, 324 bp, Artificial Sequence
  Synthetic Polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggagggaccaaagtggatatcaaacgt

SEQ ID NO: 34 - protein, 10 aa, Artificial Sequence
  Synthetic Polypeptide
GYSFTNFWIS

SEQ ID NO: 35 - protein, 17 aa, Artificial Sequence
  Synthetic Polypeptide
RVDPGYSYSTYSPSFQG

SEQ ID NO: 36 - protein, 12 aa, Artificial Sequence
  Synthetic Polypeptide
VQYSGYYDWFDP

SEQ ID NO: 37 - DNA, 30 bp, Artificial Sequence
  Synthetic Polynucleotide
ggatacagcttcaccaacttctggatcagc

SEQ ID NO: 38 - DNA, 51 bp, Artificial Sequence
  Synthetic Polynucleotide
agggttgatcctggctactcttatagcacctacagcccgtccttccaaggc

SEQ ID NO: 39 - DNA, 36 bp, Artificial Sequence
  Synthetic Polynucleotide
gtacaatatagtggctactatgactggttcgacccc

SEQ ID NO: 40 - protein, 13 aa, Artificial Sequence
  Synthetic Polypeptide
SGSSSNIGSNTVN

SEQ ID NO: 41 - protein, 7 aa, Artificial Sequence
  Synthetic Polypeptide
SNNQRPS

SEQ ID NO: 42 - protein, 11 aa, Artificial Sequence
  Synthetic Polypeptide
AAWDDSLNGWV

SEQ ID NO: 43 - DNA, 39 bp, Artificial Sequence
  Synthetic Polynucleotide
tctggaagcagctccaacatcggaagtaatactgtaaac

SEQ ID NO: 44 - DNA, 21 bp, Artificial Sequence
  Synthetic Polynucleotide
agtaataatcagcggccctca

SEQ ID NO: 45 - DNA, 33 bp, Artificial Sequence
  Synthetic Polynucleotide
gcagcatgggatgacagcctgaatggttgggtg

SEQ ID NO: 46 - protein, 121 aa, Artificial Sequence
  Synthetic Polypeptide
QMQLVQSGAEVKEPGESLRISCKGSGYSFTNFWISWVRQMPGKGLEWMGRVDPGYSYSTYSPSFQGHVTISADKSTSTAYLQWNSLKASDTAMYYCARVQYSGYYDWFDPWGQGTLVTVSS

SEQ ID NO: 47 - DNA, 363 bp, Artificial Sequence
  Synthetic Polynucleotide
cagatgcagctggtgcagtccggagcagaggtgaaagagcccggggagtctctgaggatctcctgtaagggttctggatacagcttcaccaacttctggatcagctgggtgcgccagatgcccgggaaaggcctggagtggatggggagggttgatcctggctactcttatagcacctacagcccgtccttccaaggccacgtcaccatctcagctgacaagtctaccagcactgcctacctgcagtggaacagcctgaaggcctcggacaccgccatgtattactgtgcgagagtacaatatagtggctactatgactggttcgacccctggggccagggaaccctggtcaccgtctcctca

SEQ ID NO: 48 - protein, 111 aa, Artificial Sequence
  Synthetic Polypeptide
QAVVTQPPSASGTPGQRVTISCSGSSSNIGSNTVNWYQQVPGTAPKLLIYSNNQRPSGVPDRFSGSKSGTSASLAISGLQSEDEADYYCAAWDDSLNGWVFGGGTKLTVLG

SEQ ID NO: 49 - DNA, 333 bp, Artificial Sequence
  Synthetic Polynucleotide
caggctgtggtgactcagccaccctcagcgtctgggacccccgggcagagggtcaccatctcttgttctggaagcagctccaacatcggaagtaatactgtaaactggtaccagcaggtcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttgggtgttcggcggagggaccaagctgaccgtcctaggt

SEQ ID NO: 50 - protein, 10 aa, Artificial Sequence
  Synthetic Polypeptide
GYNFSNKWIG

SEQ ID NO: 51 - protein, 17 aa, Artificial Sequence
  Synthetic Polypeptide
IIYPGYSDITYSPSFQG

SEQ ID NO: 52 - protein, 9 aa, Artificial Sequence
  Synthetic Polypeptide
HTALAGFDY

SEQ ID NO: 53 - DNA, 30 bp, Artificial Sequence
  Synthetic Polynucleotide
ggctacaactttagcaacaagtggatcggc

SEQ ID NO: 54 - DNA, 51 bp, Artificial Sequence
  Synthetic Polynucleotide
atcatctatcccggttactcggacatcacctacagcccgtccttccaaggc

SEQ ID NO: 55 - DNA, 27 bp, Artificial Sequence
  Synthetic Polynucleotide
cacacagctttggccggctttgactac

SEQ ID NO: 56 - protein, 11 aa, Artificial Sequence
  Synthetic Polypeptide
RASQNINKWLA

SEQ ID NO: 57 - protein, 7 aa, Artificial Sequence
  Synthetic Polypeptide
KASSLES

SEQ ID NO: 58 - protein, 8 aa, Artificial Sequence
  Synthetic Polypeptide
QQYNSYAT

SEQ ID NO: 59 - DNA, 33 bp, Artificial Sequence
  Synthetic Polynucleotide
cgggccagtcagaatatcaataagtggctggcc

SEQ ID NO: 60 - DNA, 21 bp, Artificial Sequence
  Synthetic Polynucleotide
aaggcgtctagtttagaaagt

SEQ ID NO: 61 - DNA, 24 bp, Artificial Sequence
  Synthetic Polynucleotide
caacaatataatagttatgcgacg

SEQ ID NO: 62 - protein, 118 aa, Artificial Sequence
  Synthetic Polypeptide
QVQLVQSGAEVKKPGESLKISCKGSGYNFSNKWIGWVRQLPGRGLEWIAIIYPGYSDITYSPSFQGRVTISADTSINTAYLHWHSLKASDTAMYYCVRHTALAGFDYWGLGTLVTVSS

SEQ ID NO: 63 - DNA, 354 bp, Artificial Sequence
  Synthetic Polynucleotide
caggtgcagctggtgcagtctggagcagaggtgaaaaagcccggagagtctctgaagatctcctgtaagggttctggctacaactttagcaacaagtggatcggctgggtgcgccaattgcccgggagaggcctggagtggatagcaatcatctatcccggttactcggacatcacctacagcccgtccttccaaggccgcgtcaccatctccgccgacacgtccattaacaccgcctacctgcactggcacagcctgaaggcctcggacaccgccatgtattattgtgtgcgacacacagctttggccggctttgactactggggcctgggcaccctggtcaccgtctcctca

SEQ ID NO: 64 - protein, 107 aa, Artificial Sequence
  Synthetic Polypeptide
DIQMTQSPSTLSASVGDRVTITCRASQNINKWLAWYQQRPGKAPQLLIYKASSLESGVPSRFSGSGSGTEYTLTISSLQPDDFATYYCQQYNSYATFGQGTKVEIKR

SEQ ID NO: 65 - DNA, 321 bp, Artificial Sequence
  Synthetic Polynucleotide
gacatccagatgacccagtctccttccaccctgtctgcatctgtaggagacagagtcacaatcacttgccgggccagtcagaatatcaataagtggctggcctggtatcagcagagaccagggaaagcccctcagctcctgatctataaggcgtctagtttagaaagtggggtcccatctaggttcagcggcagtggatctgggacagaatacactctcaccatcagcagcctgcagcctgatgattttgcaacttattactgccaacaatataatagttatgcgacgttcggccaagggaccaaggtggaaatcaaacgt

SEQ ID NO: 66 - protein, 10 aa, Artificial Sequence
  Synthetic Polypeptide
GFTFDDYGMS

SEQ ID NO: 67 - protein, 17 aa, Artificial Sequence
  Synthetic Polypeptide
GINWNGGSTGYADSVRG

SEQ ID NO: 68 - protein, 12 aa, Artificial Sequence
  Synthetic Polypeptide
ERGYGYHDPHDY

SEQ ID NO: 69 - DNA, 30 bp, Artificial Sequence
  Synthetic Polynucleotide
gggttcacctttgatgattatggcatgagc

SEQ ID NO: 70 - DNA, 51 bp, Artificial Sequence
  Synthetic Polynucleotide
ggtattaattggaatggtggtagcacaggttatgcagactctgtgaggggc

SEQ ID NO: 71 - DNA, 36 bp, Artificial Sequence
  Synthetic Polynucleotide
gagcgtggctacgggtaccatgatccccatgactac

SEQ ID NO: 72 - protein, 11 aa, Artificial Sequence
  Synthetic Polypeptide
GRNNIGSKSVH

SEQ ID NO: 73 - protein, 7 aa, Artificial Sequence
  Synthetic Polypeptide
DDSDRPS

SEQ ID NO: 74 - protein, 11 aa, Artificial Sequence
  Synthetic Polypeptide
QVWDSSSDHVV

SEQ ID NO: 75 - DNA, 33 bp, Artificial Sequence
  Synthetic Polynucleotide
gggagaaacaacattggaagtaaaagtgtgcac

SEQ ID NO: 76 - DNA, 21 bp, Artificial Sequence
  Synthetic polynucleotide
gatgatagcgaccggccctca

SEQ ID NO: 77 - DNA, 33 bp, Artificial Sequence
  Synthetic Polynucleotide
caggtgtgggatagtagtagtgatcatgtggta

SEQ ID NO: 78 - protein, 121 aa, Artificial Sequence
  Synthetic Polypeptide
EVQLVQSGGGVVRPGGSLRLSCAASGFTFDDYGMSWVRQAPGKGLEWVSGINWNGGSTGYADSVRGRFTISRDNAKNSLYLQMNSLRAEDTALYYCARERGYGYHDPHDYWGQGTLVTVSS

SEQ ID NO: 79 - DNA, 363 bp, Artificial Sequence
  Synthetic Polynucleotide
gaagtgcagctggtgcagtctgggggaggtgtggtacggcctggggggtccctgagactctcctgtgcagcctctgggttcacctttgatgattatggcatgagctgggtccgccaagctccagggaaggggctggagtgggtctctggtattaattggaatggtggtagcacaggttatgcagactctgtgaggggccgattcaccatctccagagacaacgccaagaactccctgtatctgcaaatgaacagtctgagagccgaggacacggccttgtattactgtgcgagagagcgtggctacgggtaccatgatccccatgactactggggccaaggcaccctggtgaccgtctcctca

SEQ ID NO: 80 - protein, 109 aa, Artificial Sequence
  Synthetic Polypeptide
QSVVTQPPSVSVAPGKTARITCGRNNIGSKSVHWYQQKPGQAPVLVVYDDSDRPSGIPERFSGSNSGNTATLTISRVEAGDEADYYCQVWDSSSDHVVFGGGTKLTVLG

SEQ ID NO: 81 - DNA, 327 bp, Artificial Sequence
  Synthetic Polynucleotide
cagtctgtcgtgacgcagccgccctcggtgtcagtggccccaggaaagacggccaggattacctgtgggagaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatgtggtattcggcggagggaccaagctgaccgtcctaggt

SEQ ID NO: 82 - protein, 10 aa, Artificial Sequence
  Synthetic Polypeptide
GFSVSGTYMG

SEQ ID NO: 83 - protein, 16 aa, Artificial Sequence
  Synthetic Polypeptide
LLYSGGGTYHPASLQG

SEQ ID NO: 84 - protein, 10 aa, Artificial Sequence
  Synthetic Polypeptide
GGAGGGHFDS

SEQ ID NO: 85 - DNA, 30 bp, Artificial Sequence
  Synthetic Polynucleotide
gggttctccgtcagtggcacctacatgggc

SEQ ID NO: 86 - DNA, 48 bp, Artificial Sequence
  Synthetic Polynucleotide
cttctttatagtggtggcggcacataccacccagcgtccctgcagggc

SEQ ID NO: 87 - DNA, 29 bp, Artificial Sequence
  Synthetic Polynucleotide
gaggggcaggaggtggccactttgactcc

SEQ ID NO: 88 - protein, 14 aa, Artificial Sequence
  Synthetic Polypeptide
TGSSSNIGAGYDVH

SEQ ID NO: 89 - protein, 7 aa, Artificial Sequence
  Synthetic Polypeptide
GNSNRPS

SEQ ID NO: 90 - protein, 11 aa, Artificial Sequence
  Synthetic Polypeptide
AAWDDSLNGYV

SEQ ID NO: 91 - DNA, 42 bp, Artificial Sequence
  Synthetic Polynucleotide
actgggagcagctccaacatcggggcaggttatgatgtacac

SEQ ID NO: 92 - DNA, 21 bp, Artificial Sequence
  Synthetic Polynucleotide
ggtaacagcaatcggccctca

SEQ ID NO: 93 - DNA, 33 bp, Artificial Sequence
  Synthetic Polynucleotide
gcagcatgggatgacagcctgaatggttatgtc

SEQ ID NO: 94 - protein, 118 aa, Artificial Sequence
  Synthetic Polypeptide
EVQLVETGGGLLQPGGSLRLSCAASGFSVSGTYMGWVRQAPGKGLEWVALLYSGGGTYHPASLQGRFIVSRDSSKNMVYLQMNSLKAEDTAVYYCAKGGAGGGHFDSWGQGTLVTVSS

SEQ ID NO: 95 - DNA, 354 bp, Artificial Sequence
  Synthetic Polynucleotide
gaggtgcagctggtggagaccggaggaggcttgctccagccgggggggtccctcagactctcctgtgcagcctctgggttctccgtcagtggcacctacatgggctgggtccgccaggctccagggaagggactggagtgggtcgcacttctttatagtggtggcggcacataccacccagcgtccctgcagggccgattcatcgtctccagagacagctccaagaatatggtctatcttcaaatgaatagcctgaaagccgaggacacggccgtctattactgtgcgaaaggaggggcaggaggtggccactttgactcctggggccaaggcaccctggtgaccgtctcctca

SEQ ID NO: 96 - protein, 112 aa, Artificial Sequence
  Synthetic Polypeptide
QSVLTQPPSVSGAPGQRVTISCTGSSSNIGAGYDVHWYQQLPGTAPKLLIYGNSNRPSGVPDRFSGSKSGTSASLAISGLQSEDEADYYCAAWDDSLNGYVFGTGTKLTVLG

SEQ ID NO: 97 - DNA, 336 bp, Artificial Sequence
  Synthetic Polynucleotide
cagtctgtgttgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctgaccgtcctaggt

SEQ ID NO: 98 - protein, 105 aa, Artificial Sequence
  Synthetic Polypeptide
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 99 - protein, 328 aa, Artificial Sequence
  Synthetic Polypeptide
TKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 232 aa, Artificial Sequence
  Synthetic Polypeptide
MGWSCIILFLVATATGQAVVTQPPSASGTPGQRVTISCSGSSSNIGSNTVNWYQQVPGTAPKLLIYSNNQRPSGVPDRFSGSKSGTSASLAISGLQSEDEADYYCAAWDDSLNGWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 101 - protein, 467 aa, Artificial Sequence
  Synthetic Polypeptide
MGWSCIILFLVATATGQMQLVQSGAEVKEPGESLRISCKGSGYSFTNFWISWVRQMPGKGLEWMGRVDPGYSYSTYSPSFQGHVTISADKSTSTAYLQWNSLKASDTAMYYCARVQYSGYYDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 16 aa, Artificial Sequence
  Synthetic Polypeptide
MGWSCIILFLVATATG